SEQUENCE LISTING (10 sequences)

SEQ ID NO: 1 - DNA, 44 bp, ARTIFICIAL SEQUENCE
  miR122
cgcagtagagctctagtacaaacaccattgtcacactccagttt

SEQ ID NO: 2 - DNA, 45 bp, ARTIFICIAL SEQUENCE
  miR122
aactggagtgtgacaatggtgtttgtactagagctctactgcgat

SEQ ID NO: 3 - DNA, 15 bp, ARTIFICIAL SEQUENCE
  Primer
gctaagaagttccct

SEQ ID NO: 4 - RNA, 23 nt, ARTIFICIAL SEQUENCE
  miR-122 2′-OMe PS antagomir
acaaacaccauugucacacucca

SEQ ID NO: 5 - DNA, 22 bp, ARTIFICIAL SEQUENCE
  Primer
gctcttcccattgctcaagatg

SEQ ID NO: 6 - DNA, 20 bp, ARTIFICIAL SEQUENCE
  Primer
gtatgtaacaacagcatgtg

SEQ ID NO: 7 - DNA, 23 bp, ARTIFICIAL SEQUENCE
  Primer
cgggagagccatagtggtctgcg

SEQ ID NO: 8 - DNA, 25 bp, ARTIFICIAL SEQUENCE
  Primer
ctcgcaagcaccctatcaggcagta

SEQ ID NO: 9 - DNA, 20 bp, ARTIFICIAL SEQUENCE
  Primer
tgcaccaccaactgcttagc

SEQ ID NO: 10 - DNA, 21 bp, ARTIFICIAL SEQUENCE
  Primer
ggcatggactgtggtcatgag

(19) United States
(12) Patent Application Publication
(10) Pub. No.: US 2013/0005759 A1
(43) Pub. Date: Jan. 3, 2013

(54) SMALL MOLECULE MODIFIERS OF MICRORNA MIR-122

(75) Inventors: Alexander Deiters, Raleigh, NC (US); Douglas D. Young, Williamsburg, VA (US)

(73) Assignee: North Carolina State University

(21) Appl. No.: 13/574,443

(22) PCT Filed: Jan. 21, 2011
(86) PCT No.: PCT/US11/21976

§ 371 Date: Sep. 7, 2012

Related U.S. Application Data
(60) Provisional application No. 61/297,148, filed on Jan. 21, 2010.
(60) Provisional application No. 61/297,920, filed on Jan. 25, 2010.

(51) Int. Cl. A61K 31/167; C07C 233/65; A61P 31/14; C12N 5/071; A61K 31/473; C07D 401/12; A61K 31/4709; C07D 215/36; A61K 31/47; C07D 221/14; C12Q 1/68; A61P 35/00

(52) U.S. Cl. 514/296; 435/6.13; 564/184; 514/617; 435/375; 564/169; 514/621; 564/166; 514/619; 564/176; 514/622; 546/153; 514/312; 546/165; 514/314; 514/311; 546/100

(57) ABSTRACT

MicroRNAs are a class of endogenous regulators of gene function. Aberrant regulation of microRNAs has been linked to various human diseases, most importantly cancer. Small molecule intervention of microRNA misregulation has the potential to provide new therapeutic approaches to such diseases. microRNA miR-122 is the most abundant microRNA in the liver and is involved in hepatocellular carcinoma development and hepatitis C virus (HCV) infection. Small molecule inhibitors and activators of the microRNA miR-122 are described, and methods for their identification are reported. These small molecule inhibitors reduce viral replication in liver cells and thus represent a new approach to the treatment of HCV infections. Moreover, small molecule activation of miR-122 in liver cancer cells selectively induced apoptosis through caspase activation, and thus has implications in cancer chemotherapy.

### FIELD OF THE INVENTION

The present invention relates to small molecule inhibitors and small molecule activators of miR-122, use of these small molecules for treating or preventing diseases and disorders associated with miR-122, including those of the liver, and a method for identifying inhibitors and activators of miR-122.

### BACKGROUND OF THE INVENTION

MicroRNAs (miRNAs) are single-stranded noncoding RNAs of 21-23 nucleotides. They are a class of gene regulators that function by binding the 3′ untranslated regions of specific target messenger RNAs leading to gene inactivation by repression of mRNA transcription or induction of mRNA degradation.¹ MicroRNAs are transcribed from the genome and undergo several post-transcriptional processing steps via a dedicated microRNA pathway. It is estimated that 1000 miRNAs exist in humans, controlling approximately 30% of all genes, thus being involved in almost every genetic pathway and many human pathologies, e.g. cancer, heart disease, and viral infection.²

MicroRNA miR-122 is present in the liver and is the most abundant miRNA in the liver.³ It was discovered that miR-122 is greatly down-regulated in hepatocellular carcinoma (HCC). Identified targets of miR-122 in primary liver carcinomas and the HCC cell lines Hep3B and HepG2 are cyclin G1 (CCNG1) and Bcl-w, an anti-apoptotic Bcl-2 family member.⁴ HCC is a primary cancer of the liver, and it is the third largest cause of cancer-related death behind only lung and colon cancers.⁵ Treatment options of HCC and prognosis are usually poor (with a median survival time of 3 to 6 months), as only 10-20% of hepatocellular carcinomas can be removed completely using surgery.⁵ Transfection of miR-122 into cancer cells induced apoptosis and led to reduced cell viability. Consequentially, the induction of apoptosis in malignant hepatocytes through the activation of miR-122 expression represents a potential treatment for HCC.

The liver-specific miR-122 is also necessary for hepatitis C virus (HCV) replication and infectious virus production through interaction with the viral genome.⁶ HCV infection is one of the major causes of chronic liver disease, including cirrhosis and liver cancer and is therefore the most common indication for liver transplantation. The role of miR-122 in HCV replication suggests that it could be a used for antiviral therapy, since knock-down of miR-122 results in a dramatic loss of HCV RNA in human liver cells,⁶ without any toxic effects in mice and primates.^{7,8}

The inventors' discovery of small molecule modifiers of miR-122 validates miRNA as a therapeutic target. Recently, the first small molecule inhibitors of miRNA function, specifically miR-21 function was reported.⁹ These compounds displayed specificity for miR-21 and induced a reduction of both mature-miR-21 and primary-miR-21 levels. Additionally, the small molecule enoxacin has been demonstrated to be a general activator of both the siRNA and miRNA pathways,¹⁰ and while not being bound by any theory, this may be through promoting the processing and loading of siRNAs/miRNAs into RNA silencing complexes (RISCs) by facilitating the interaction between transactivating response (TAR) RNA-binding protein and RNAs.

### SUMMARY OF THE INVENTION

Small molecule modifiers of the liver-specific microRNA miR-122, have been identified and it was demonstrated that small molecule inhibitors and activators of miR-122 function have therapeutic potential. In order to identify inhibitors and activators of miR-122a reporter system for identifying the activation or inhibition of miR-122 function was established and used for the screening of molecules.

These small molecule miRNA activators and inhibitors represent unique tools for the elucidation of miR-122 biogenesis and regulation in healthy liver tissue and in other tissues where miR-122 is present, and have the potential to provide new targets and lead structures for the development of therapeutics.

An aspect of the invention relates to an assay for identifying an agent that modulates the function of miR-122.

Another aspect of the invention relates to an assay for identifying an agent that inhibits the function of miR-122.

Yet another aspect of the invention related to an assay for identifying an agent that activates the function of miR-122.

Still another aspect of the invention relates to a method for preventing or treating a disease or condition of the liver.

Another aspect of the invention relates to the treatment of hepatocellular carcinoma using a compound that modulates the activity of miR-122.

Another aspect of the invention relates to the treatment of hepatocellular carcinoma using a compound that activates the activity of miR-122.

Another aspect of the invention is a method for treating a subject infected with hepatitis C virus using a compound that modulates the activity of miR-122.

Another aspect of the invention is a method for treating a subject infected with hepatitis C virus using a compound that inhibits the activity of miR-122.

Another aspect of the invention relates to compounds that modulate, inhibit or activate the function of miR-122.

Yet another aspect of the invention relates to compounds and compositions that can be used in the prevention or treatment of diseases of the liver or other organ where miR-122 is present.

### BRIEF DESCRIPTION OF THE FIGURES

FIG. 1 (a) shows that in the microRNA miR-122 assay the developed luciferase reporter can detect the presence of a functional mature miR-122 through repression of the luciferase signal.

FIG. 1 (b) shows that in the microRNA miR-122 assay small molecules are assayed for their ability to alleviate the suppression of luciferase expression by inhibition of miR-122, thus inducing luciferase expression.

FIG. 2 shows validation of the psiCHECK-miR122 vector for the detection of miR-122 in both HeLa and Huh7 cell lines. The error bars represent the standard deviation from three independent experiments.

FIG. 3 shows validation of small molecule hits 1 and 2 from the NCI Diversity Set screen. All experiments were conducted in triplicate, and a significant increase in Renilla luciferase expression is observed when Huh7 cells are treated with the antagomir or compound 1 and 2 (10 μM). The relative luciferase units were normalized to the corresponding DMSO signals. The error bars represent the standard deviation from three independent experiments.

FIG. 4 shows determination of miRNA specificity of compounds 1 and 2 using the miR-21 assay of this invention. (10 μM). Neither compound 1 nor 2 demonstrated an upregulation of luciferase levels, suggesting that they are not general inhibitors of the miRNA pathway. The diazobenzene A (10 μM) is our previously discovered miR-21 inhibitor⁹. The error bars represent the standard deviation from three independent experiments.

FIG. 5 shows luciferase assay dose response curves for compounds 1-3. The insert shows only the activator 3. All assays were conducted in triplicate and normalized to a DMSO control.

FIG. 6 shows RT PCR quantification of miR-122 and pri-miR-122 in Huh7 cells, and miR-21 in HeLa cells exposed to A) inhibitors 1 and 2 (10 μM), and B) activator 3 (10 μM). All experiments were conducted in triplicate, and the data was normalized to a DMSO control.

FIG. 7 shows effects of miR-122 inhibitors 1 and 2 on HCV replication in Huh7 cells. Both compounds led to a substantial reduction of viral RNA in infected cells. Control experiments contain only 1% DMSO and no compound. All experiments were conducted in triplicate.

FIG. 8 show effects of the miR-122 activator 3 on HepG2 cell viability. FIG. 8 A) shows that an increased caspase 3/7 level is observed in HepG2 cells due to activation of the pro-apoptotic miR-122. This increase is more pronounced in HepG2 than Huh7 cells, due to the different basal miR-122 levels in both cell lines. FIG. 8 B) shows the increase in caspase-3 activity leads to loss of cellular viability in HepG2 cells exposed to activator 3 (10 μM) relative to Huh7 cells. Control experiments contain only 1% DMSO and no compound.

FIG. 9 illustrates the psiCHECK™-2 Vector.

FIG. 10 shows small molecule inhibitor 1 of miR-122 and analogs of inhibitor 1. The numbers in parentheses represent relative luciferase units (RLU) normalized to a DMSO control, and the standard deviation is derived from three independent assays.

FIG. 11 shows small molecule inhibitor 2 of miR-122 and analogs of inhibitor 2. The numbers in parentheses represent relative luciferase units (RLU) normalized to a DMSO control, and the standard deviation is derived from three independent assays.

FIG. 12 shows small molecule inhibitor 3 of miR-122 and analogs of inhibitor 3. The numbers in parentheses represent relative luciferase units (RLU) normalized to a DMSO control, and the standard deviation is derived from three independent assays

### DEFINITIONS

Listed below are definitions, which apply to the terms as they are used throughout the specification and claims (unless they are limited in specific instances). Unless defined otherwise, all technical and scientific terms used herein have the same meaning as commonly understood by one of ordinary skill in the art to which this invention belongs.

As used herein, “activator” means a small molecule that enhances microRNA function. An example of an activator is a small molecule that increases the level of mature miRNA.

As used herein, “amelioration of the symptoms” of a particular disorder by administration of a particular compound or composition refers to any lessening, whether permanent or temporary, lasting or transient of a symptom that can be attributed to or associated with administration of the compound or composition.

An “effective amount” as used herein would also include an amount of a compound or composition sufficient to prevent, ameliorate or delay the development of a symptom of the disease or condition, alter the course of a symptom disease (for example but not limited to, slow the progression of a symptom of the disease), or reverse a symptom of the disease. Such amount may be administered as a single dosage or may be administered according to a regimen, whereby it is effective.

As used herein, “inhibitor” means a small molecule that inhibits microRNA function. An example of an inhibitor is a small molecule that decreases the level of mature miRNA.

As used herein, “luminescence” refers to the detectable EM radiation, generally, UV, IR or visible EM radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules, or synthetic versions or analogs thereof as substrates and/or enzymes.

As used herein “modifier” means a small molecule that modifies microRNA function. An example of a modifier is a small molecule that either increases or decreases the level of mature miRNA.

As used herein, “modulating” or “modulator” is a compound that alters the function, reaction or activity of a biological molecule.

The terms “patient”, “subject” and “individual” are used interchangeably herein, and refer to human and non-human animals and transgenic species thereof to whom administration of the compound or composition is provided. The term “non-human animals” includes all vertebrates, e.g. mammals, such as non-human primates, sheep, dog, cow, rodent (e.g. mouse or rat), guinea pig, goat, pig, cat, rabbits, and non-mammals such as chickens, amphibians, reptiles etc. In one embodiment, the subject is human. In another embodiment, the subject is an experimental animal or animal substitute as a disease model.

As used herein the term “pharmaceutically acceptable” is meant that the carrier, diluent, excipients, and/or salt must be compatible with the other ingredients of the composition, and not deleterious to the recipient thereof. Each carrier must be “acceptable” in the sense of being compatible with the other ingredients of the formulation.

“Pharmaceutically acceptable” also means that the compositions, or dosage forms are within the scope of sound medical judgment, suitable for use for an animal or human without excessive toxicity, irritation, allergic response, or other problem or complication, commensurate with a reasonable benefit/risk ratio.

The term “pharmaceutically acceptable salts” refers to the non-toxic, inorganic and organic acid addition salts and base addition salts of compounds of the present invention.

“Prevention” as used herein, refers to delaying, slowing, inhibiting, reducing or ameliorating the onset of disease.

As used herein the term “small molecule” is a molecule with a low molecular weight, typically smaller than 1000 Da.

As used herein, the term “solvate” preferably refers to a compound formed by the interaction of a solute (in this invention, a compound that is an activator, inhibitor, or modulator of miR-122) and a solvent. Such solvents for the purpose of the invention may not interfere with the biological activity of the solute.

As used herein, the term “stereoisomer” is a general term used for all isomers of individual compounds that differ only in the orientation of their atoms in space. The term stereoisomer includes mirror image isomers (enantiomers), mixtures of mirror image isomers (racemates, racemic mixtures), geometric (cis/trans or E/Z) isomers, and isomers of compounds with more than one chiral center that are not mirror images of one another (diastereoisomers). The compounds of the present invention may have asymmetric centers and occur as racemates, racemic mixtures, individual diastereoisomers, or enantiomers, or may exist as geometric isomers, with all isomeric forms of said compounds being included in the present invention.

As used herein, the term “tautomer” refers to the coexistence of two (or more) compounds that differ from each other only in the position of one (or more) mobile atoms and in electron distribution, for example, keto-enol tautomers.

As used herein, the terms “treatment” and “therapy” and the like refer to alleviate, slow the progression, prophylaxis, attenuation or cure of existing disease. “Treatment” of a subject includes the application or administration of a compound or composition to a subject, or application or administration of a compound or composition to a cell or tissue from a subject who has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition.

### DETAILED DESCRIPTION OF THE INVENTION

The present invention relates, in part, to a screen for identifying small molecule modifiers of the function of miR-122.

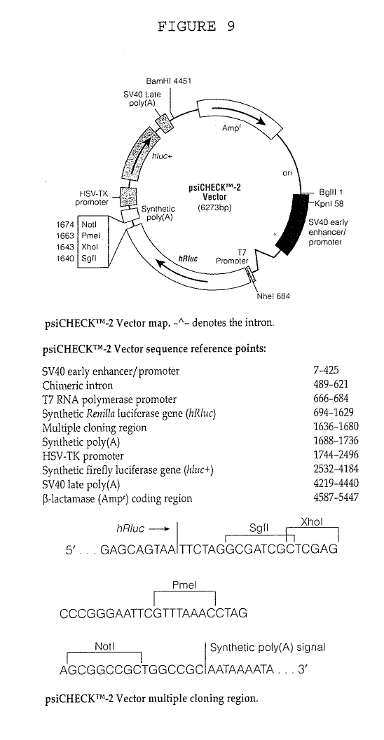

A miRNA small molecule modifier screen for miR-122 was developed based on the psiCHECK-2 (Promega) reporter plasmid (FIG. 9). This construct expresses both Renilla luciferase as well as firefly luciferase, allowing for the normalization of the signal to account for differential cellular viability and variability in transfection efficiency. The miR-122 target sequence was inserted downstream of the Renilla luciferase gene, between the PmeI and SgfI restriction sites. In another aspect of the invention, plasmids expressing luciferase or another reporter gene such as for example, fluorescent reporters (GFP and variants thereof, DsRed, etc.) or beta-galactosidase can be used to screen for small molecule modifiers of miR-122. The mi-122 target sequence may be upstream or downstream of the reporter gene. Multiple miR-122 target sites can also be inserted into or be present in the plasmid.

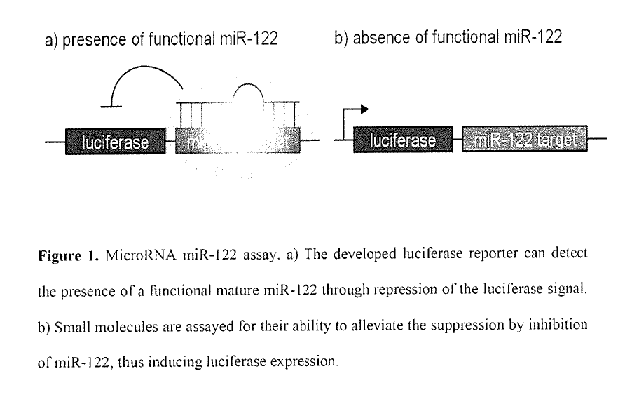

The presence of mature miR-122 will lead to a decrease in the Renilla luciferase signal (FIG. 1a). The ability to detect endogenous miR-122 was validated by transfecting the generated psiCHECK-miR122 construct into Huh7 and HeLa cells. After 48 hour incubation, the cells were assayed using a Dual Luciferase Assay Kit (Promega). In another embodiment, the cells can be incubated for about 24-96 hours. The Dual Luciferase Assay Kit provides a means to measure the amount of the luciferases by generating a luminescent signal. Using this assay the luminescence from the firefly luciferase reaction may be quenched while the luminescent reaction of Renilla luciferase can be activated. Other assays and procedures that can determine the amount of the one or more reporter genes or miR-122 expression, activity or function can be used. An example of another assay is Dual-Glo Assay (Promega).

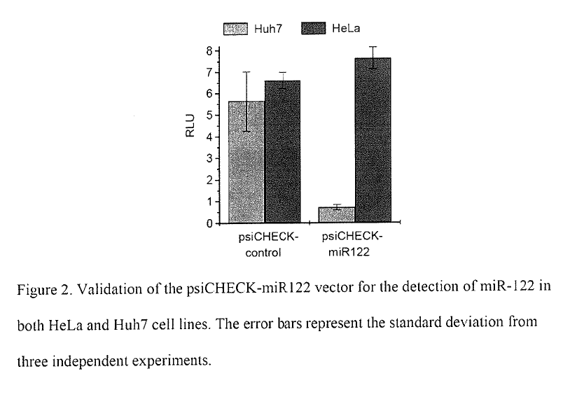

Huh7 cells have previously been demonstrated to express high levels of miR-122,⁷ whereas miR-122 is not expressed in HeLa cells.¹¹ Huh7 cells demonstrate a significant decrease in the relative Renilla luciferase signal due to the presence of miR-122. HeLa cells do not express miR-122,¹⁸ which is confirmed by comparable signals for Renilla and firefly luciferase in the psiCHECK-miR122 and the psiCHECK-control vector The psiCHECK-miR122 reporter verified these results by displaying a >15-fold reduced luciferase signal in Huh7 cells, in contrast to HeLa cells (FIG. 2), and thus is a cellular sensor for miR-122 expression. The luciferase signal is readily restored upon the co-transfection with a miR-122 antagomir, suggesting that the reporter can be employed in the discovery of miR-122 inhibitors (FIG. 1b). Moreover, a psiCHECK-control reporter (containing an empty multi-cloning site) is not affected. The signal-to-background ratio is 9.0 and the statistical parameter Z′ is 0.66, demonstrating a robust assay.¹² The variation between plates and from day-to-day is around 10%, and therefore fairly small.

The ability of the psiCHECK-miR122 reporter plasmid to detect endogenous miRNA levels in Huh7 cells has been validated.

The psiCHECK-miR122 vector was then employed in a small molecule screen in Huh7 cells to discover modifiers of miR-122 function. Other cells that express miR-122 such as HepG2 and Hep3B can be used in addition to or instead of Huh7 cells. An increase in the relative Renilla luciferase signal indicates a miR-122 inhibitor, while a reduction in the luciferase signal indicates a miR-122 activator. A genus of compounds that may have activity as modifiers of miR122 function are the Diversity Set II (1364 compounds) from the NCI Developmental Therapeutics Program was screened using Huh7 cells containing the psiCHECK-miR122 reporter. Cells were exposed to the small molecules and the relative luciferase signal was measured after 48 hours using a Dual Luciferase Assay Kit (Promega). In another embodiment the cells can be exposed to the small molecules for about 24-96 hours. As explained above, other assays and procedures that can determine the amount of the one or more reporter genes or miR-122 expression, activity or function can be used.

A genus of compounds that may have activity as modifiers of miR-122 function are

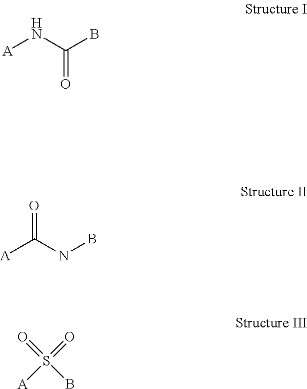

Where A and B are independently selected from unsubstituted and substituted alkyl, alkenyl, alkynyl, cycloalkyl, alkoxy, acyl, aryl, aryloxy, heterocyclyl, heteroaryl, aralkyl, NQ, and amino. Unless stated otherwise, these groups can be unsubstituted or substituted by one or more identical or different substituents. The substituents can be present at one or more positions provided that a stable molecule results.

The term “alkyl” whether used alone or as part of a substituent group, refers to the radical of saturated aliphatic groups, including straight or branched-chain containing from 1 to 10 carbon atoms. Furthermore, unless stated otherwise, the term “alkyl” includes unsubstituted as well as substituted alkyl. Suitable alkyl residues contain from 1 to 6 carbon atoms, for example, from 1 to 4 carbon atoms, such as methyl, ethyl, propyl, isopropyl, n-butyl, isobutyl and t-butyl.

The term “lower alkyl” whether used alone or as part of a substituent group, refers to the radical of saturated aliphatic groups, including straight or branched-chain containing from one to six carbon atoms, such as methyl, ethyl, n-propyl, isopropyl, n-butyl, sec-butyl, isobutyl, tert-butyl, n-pentyl and n-hexyl. The term “alkenyl” refers to an unsaturated, branched, straight chain or cyclic alkyl group having from 2 to 6 carbon atoms and at least one carbon-carbon double bond. Examples of alkenyl include, but are not limited to vinyl, allyl and 2-propenyl.

The term “alkynyl” refers to an unsaturated, branched or straight chain having from 2 to 6 carbon atoms and at least one carbon-carbon triple bond (two adjacent sp carbon atoms).

The term “cycloalkyl” refers to a saturated or partially unsaturated cyclic hydrocarbon group including 1, 2 or 3 rings and including a total of 3 to 14 carbon atoms forming the rings.

The term “alkoxy” refers to the alkyl-O— wherein the term alkyl is as defined above.

The term “acyl” refers to the group —C(O)Rₐ, wherein Rₐ is alkyl, cycloalkyl, aryl, aralkyl, heteroaryl and heteroaralkyl.

The term “aryl” refers to a monocyclic or polycyclic hydrocarbon group having up to 16 ring carbon atoms, in which at least one carbocyclic ring is present that has a conjugated it electron system. Examples of aryl residues include phenyl and naphthyl.

Aryl residues can be bonded via any desired position, and in substituted aryl residues, the substituents can be located in any desired position. For example, in monosubstituted phenyl residues the substituent can be located in the 2-position, the 3-position, the 4-position, the 5-position, or the 6-position. If the phenyl group carries two substituents, they can be located in 2,3-position, 2,4-position, 2,5-position, 2,6-position, 3,4-position or 3,5-position.

The term “aryloxy” refers to the aryl-O— wherein the term aryl is as defined above.

The terms “heterocyclyl” and “heterocyclic” refer to a saturated, partially unsaturated or aromatic monocyclic or polycyclic ring system containing 3-14 ring atoms of which 1, 2, 3 or 4 are identical or different heteroatoms selected from nitrogen, oxygen and sulfur. Monocyclic heterocyclyl groups include 3-membered, 4-membered, 5-membered, 6-membered and 7-membered rings. Suitable examples of heterocyclyl include, but are not limited to, pyrrolyl, imidazolyl, thiophenyl, pyrrolidinyl, pyridinyl, pyrazinyl, pyridazinyl, pyrimidinyl, pyrazolyl, triazolyl, tetrazolyl, piperidinyl, piperazinyl and morpholinyl. Polycyclic heterocyclyl groups can include two fused rings (bicyclic) or three fused rings (tricyclic), one of which is a 5-, 6- or 7-membered heterocyclic ring and the other is a 5-, 6- or 7-membered carbocyclic or heterocyclic ring. Exemplary bicyclic heterocyclic groups include benzoxazolyl, quinolinyl, isoquinolyl, indolyl, isoindolyl, and benzofurazanyl. Exemplary tricyclic heterocyclic groups include, but are not limited to, substituted or unsubstituted naphthofuranyl, benzoindole, pyrroloquinoline and furoquinoline. Heterocyclyl includes saturated heterocyclic ring systems, which do not contain any double bonds within the rings, as well as unsaturated heterocyclic ring systems, which contain one or more, up to 5 double bonds within the rings provided that the resulting system is stable. Unsaturated rings may be non-aromatic or aromatic.

“Heteroaryl” refers to a monocyclic or polycyclic aromatic ring comprising carbon atoms and one or more heteroatoms.

The term “aralkyl” refers to an alkyl group substituted with an aryl or heteroaryl group, wherein the terms alkyl, aryl and heteroaryl are as defined above. Exemplary aralkyl groups include —(CH₂)ₚ-phenyl, —(CH₂)ₚ-pyridyl, wherein p is an integer from 1 to 6.

The term “heteroatom” refers to nitrogen, oxygen and sulfur. It should be noted that any heteroatom with unsatisfied valences is assumed to have a hydrogen atom to satisfy the valences. The ring heteroatoms can be present in any desired number and in any position with respect to each other provided that the resulting heterocyclic system is stable and suitable as a subgroup in a drug substance.

The term “halo” or “halogen” unless otherwise stated refers to fluorine, chlorine, bromine, or iodine atom.

The term “amino” refers to the group —NH₂ which may be optionally substituted.

The groups listed above can be substituted by halogen, hydroxy, carbonyl, carboxy, alkoxy, cycloalkyl, cyano, amino, —CONH₂, imino, alkylthio, thioester, sulfonyl, nitro, haloalkyl, aralkyl, acyl, acyloxy, aryl, aryloxy, heterocyclyl, heteroaryl, —NRₓCOR_{y}, —NRₓSOR_{y}, —NRₓSO₂R_{y}, —S(O)ₘRₓ, and —S(O)ₙNRₓR_{y}, wherein Rₓ and R_{y} are independently selected from hydrogen, hydroxy, alkyl, alkoxy, alkenyl, alkynyl, cycloalkyl, aryl, aralkyl and heterocydyl; n is 0, 1 or 2 and m is 1 or 2.

Compounds 1, 2 and 3 whose structures are shown below are miRNA modifiers. The identified miRNA modifiers 1-3 are not general inhibitors or activators of the miRNA pathway, but induce inhibition or activation of miR-122 function. Compounds 1 (NSC 158959) and 2 (NSC 5476), which are inducing a 773±38% and 1251±125% increase in the relative luciferase signal are small molecule inhibitors of miR-122.

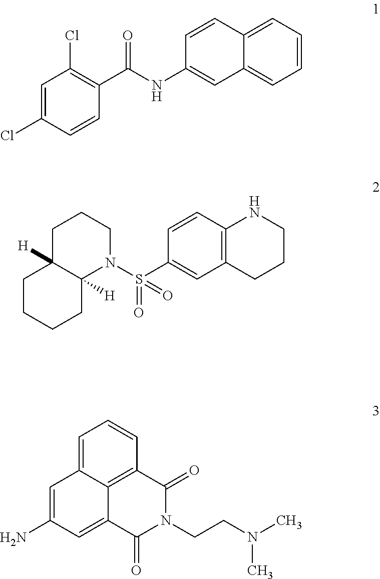 [SMILES: O=C(Nc1ccc2ccccc2c1)c1ccc(Cl)cc1Cl ; O=S(=O)(c1ccc2c(c1)CCCN2)N1CCC[C@H]2CCCC[C@@H]21 ; CN(C)CCN1C(=O)c2cccc3cc(N)cc(c23)C1=O]

The identity and purity (>95%) of compounds 1 and 2 were confirmed by NMR and mass spectrometry.

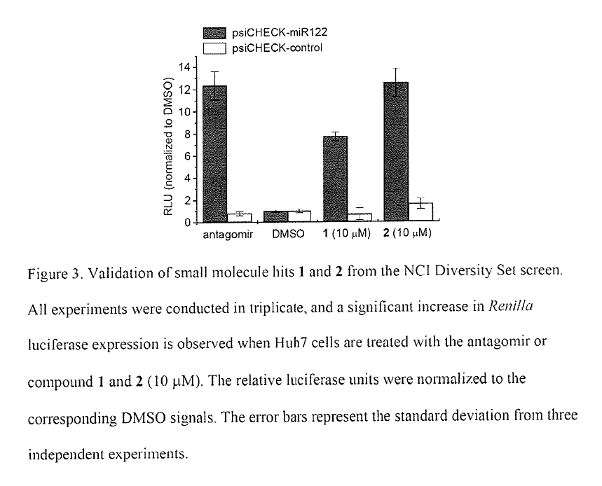

Both compounds were re-assayed in triplicate with both the psiCHECK-miR122 vector and the psiCHECK-control vector (no miRNA target sequence), confirming their activity as miR-122 inhibitors and validating that they do not increase the luciferase signal in a non-miRNA specific fashion. (FIG. 3). FIG. 3 shows validation of small molecule hits 1 and 2 from the NCI Diversity Set screen. A significant increase in Renilla luciferase expression is observed when Huh7 cells are treated with the antagomir or compound 1 or 2 (10 μM). The relative luciferase units were normalized to the corresponding DMSO signals. In the presence of the psiCHECK-control vector no statistically significant alteration in the normalized Renilla luciferase signal is observed under any of the conditions. This indicates that the increase in signal is due to the direct action of the small molecules on the miR-122 pathway. Compound 2 is capable of restoring the luciferase signal to levels comparable to the antagomir transfected cells. This confirms that compound 2 and the antagomir have similar effects on the reduction of miR-122 function in Huh7 cells.

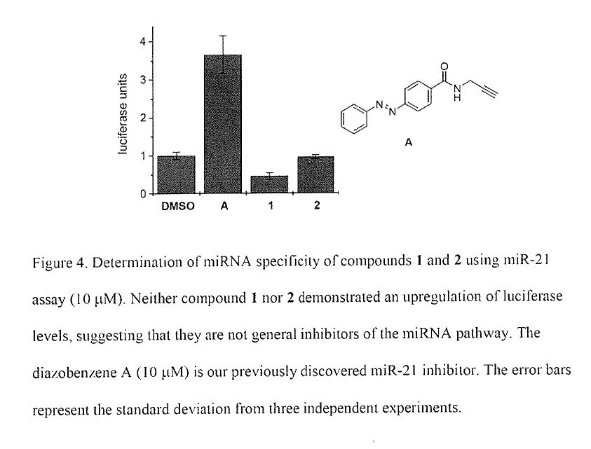

Both small molecules were also assayed with the previously described miRNA-21 reporter in HeLa cells (FIG. 4),⁹ and no activity was found suggesting that they are not general inhibitors of the miRNA pathway but display a degree of specificity for miR-122. Neither compound 1 nor 2 demonstrated an upregulation of luciferase levels, again suggesting that they are not general inhibitors of the miRNA pathway but display a degree of specificity for miR-122.

Compounds 1 and 2 did not increase the luciferase signal relative to the DMSO control, especially in comparison to our previously reported miR-21 inhibitor A⁹. This also demonstrates that compounds 1 and 2 do not inhibit miR-21 function, while they are active inhibitors of miR-122. Additionally, the miR-21 inhibitor compound A was demonstrated to have no effect in the miR-122 assay, indicating the miRNA regulation is orthogonal between the different miRNAs.

Compound 3 (NSC 308847) was found to be an activator of miR-122, inducing a 7-fold reduction of the Renilla luciferase signal (0.11±0.02 RLU). No effect of compound 3 on miR-21 controlled luciferase activity⁹ was observed in HeLa cells, indicating that compound 3 is not a general activator of the miRNA pathway, in contrast to the small molecule enoxacin,¹⁰ but shows specificity towards miR-122 activation. The molecule's identity and purity (>95%) was confirmed by both NMR and MS analysis.

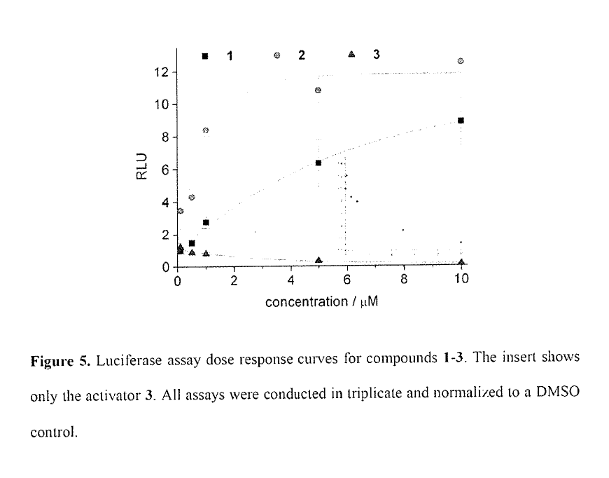

A dose dependent response was established for all three compounds which can be calculated from FIG. 5 as EC₅₀ values of 7.73±0.38 μM and 12.51±1.25 μM for the inhibitors (compounds 1 and 2, respectively.) The miR-122 activator (compound 3) displayed an IC₅₀ of 0.37±0.02 μM as calculated from FIG. 5.

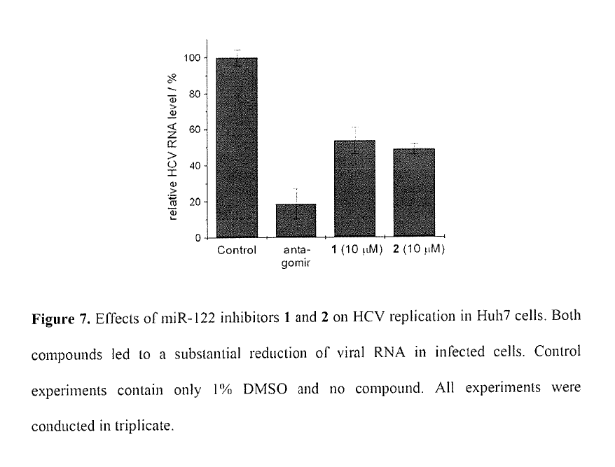

Aberrantly regulated miR-122 is involved in the development of heptocellular carcinoma (HCC). miR-122 is a cellular component required by hepatitis C virus (HCV) for viral replication. The inhibition of miR-122 by antisense oligonucleotides (antagomirs) results in a reduction of HCV replication in human liver cells (Huh7).⁶ A binding site for miR-122 was predicted to reside close to the 5′ end of the viral genome, revealing a genetic interaction between miR-122 and the viral RNA genome. Moreover, it has been shown that interferon β, currently the most common HCV therapeutic (together with interferon α), modulates the expression of several miRNAs which have target sequences in the HCV genome.¹⁵ MicroRNA miR-122 was the only down-regulated miRNA (by ˜80%) and it was demonstrated that this down-regulation plays an important role in the antiviral effects of interferon β against HCV. Due to the efficient down-regulation of miRNA-122 by the small molecule inhibitors 1 and 2, both compounds were tested for their ability to inhibit HCV replication in Huh7 cells. The pHtat2Neo/QR/KR/FV/SI plasmid (provided by Dr. Stanley Lemon of University of Texas Medical Branch (UTMB)) was used to generate genotype 1a H77c RNA, which was subsequently transfected into Huh7 cells.¹⁶ The cells were then either transfected with a miR-122 antagomir (positive control), or treated with compounds 1 and 2 (10 μM), or DMSO (negative control). After 48 hours, total RNA was isolated and HCV RNA levels were measured by quantitative RT PCR (FIG. 7). In agreement with previous reports, the antagomir reduced HCV RNA levels to 20%.⁶ Moreover, the small molecule miR-122 inhibitors 1 and 2 elicited a reduction in viral load to 48% and 47%, respectively. It is demonstrated that the small molecule miR-122 inhibitor 2 inhibits HCV replication in liver cells, thus demonstrating a fundamentally novel approach to the development of small molecule therapeutics for HCV infection.

These results are evidence that new small molecule drugs can be used for the treatment of HCV infection. Since the compounds 1 and 2 target a critical host component at the miRNA-mRNA host-pathogen interface, the virus will most likely not be able to develop a resistance to those molecules, or other miR-122 inhibitors.

Compared to healthy liver tissue, miR-122 is reduced by ˜85% in the HCC cell line Huh7 and by 99.5% in the HCC cell lines HepG2 and Hep3B,^{3,13} Thus, the screening results were also analyzed for a further reduction in the relative Renilla luciferase signal, since this would reveal molecules that would activate miR-122 function in Huh7 cells. Several compounds were identified and re-assayed with both the psiCHECK-miR122 vector and the psiCHECK-control vector.

As discussed above, miR-122 is greatly down-regulated in HCC compared to healthy liver tissue, thus inducing an upregulation of the anti-apoptotic miR-122 target Bcl-w, which subsequently leads to a deactivation of caspase-3 and an enhanced viability of cancer cells.¹³ Moreover, the small molecule activator 3 induced an increased expression of the pro-apoptotic miR-122 in the HCC cell line HepG2, leading to increased caspase expression and a reduced cell viability.

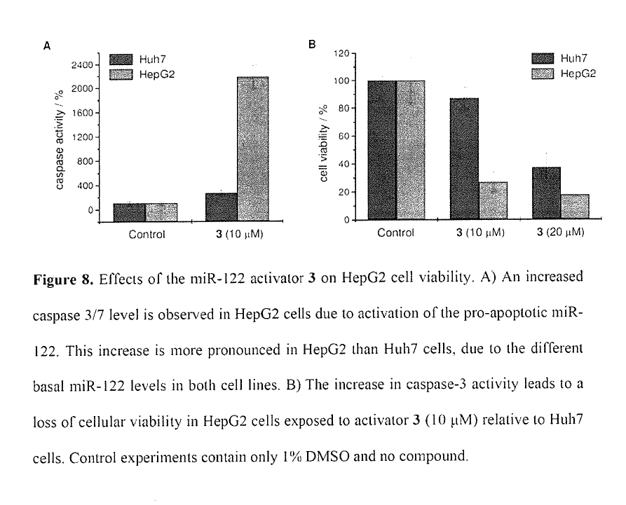

Treatment of HepG2 cells with the miR-122 activator (compound 3) led to an approximate 20-fold increase in the activity of caspase-3 and -7 (Caspase-Glo 3/7, Promega), suggesting that the increased levels of miR-122 are capable of inducing an apoptotic cascade. This enhanced caspase activity led to a reduced cell viability of ˜20% (CellTiter-Glo, Promega) (FIGS. 8A and B), exceeding previously reported reductions in cell viability due to lentiviral over-expression of miR-122 in HepG2 cells.¹³ An increased caspase 3/7 level is observed in HepG2 cells due to activation of the pro-apoptic miR-122 (FIG. 8A). This increase is more pronounced in HepG2 than Huh7 cells, due to the different basal miR-122 levels in both cell lines. FIG. 8B shows the increase in caspase-3 activity leads to a loss of cellular viability in HepG2 cells exposed to compound 3 relative to Huh7 cells. The cell viability of Huh7 cells, which express 60-fold higher miR-122 levels than HepG2 levels, is only slightly affected by exposure to compound 3, suggesting a selective apoptosis inducing effect in cells with pathologically low levels of miR-122. Thus, a small molecule activator of miR-122, like compound 3, has therapeutic relevance towards the selective treatment of HCC.

In order to validate the activity of compounds of structures A, B and C and compounds 1-3, a structure-activity relationship study was undertaken. Analogs of the compounds 1-3 were synthesized by varying the functional groups.

The compounds described below can be prepared using various procedures, some of which are depicted in the examples below. Those with skill in the art will appreciate that the specific starting compounds and reagents, such as acids, bases, solvents, reducing agents; temperature conditions etc. identified in the examples can be altered to prepare compounds encompassed by the present invention.

The analogs were screened for activity using the previously described miR-122 assay. All cells were treated with 10 μM of a compound and experiments were performed in triplicate to ensure statistical validity. The data was normalized to a 1% DMSO control and is reported in relative luciferase units (RLU). The standard deviation was calculated from three independent assays. Some analogs of miR-122 inhibitor compound 1 are:

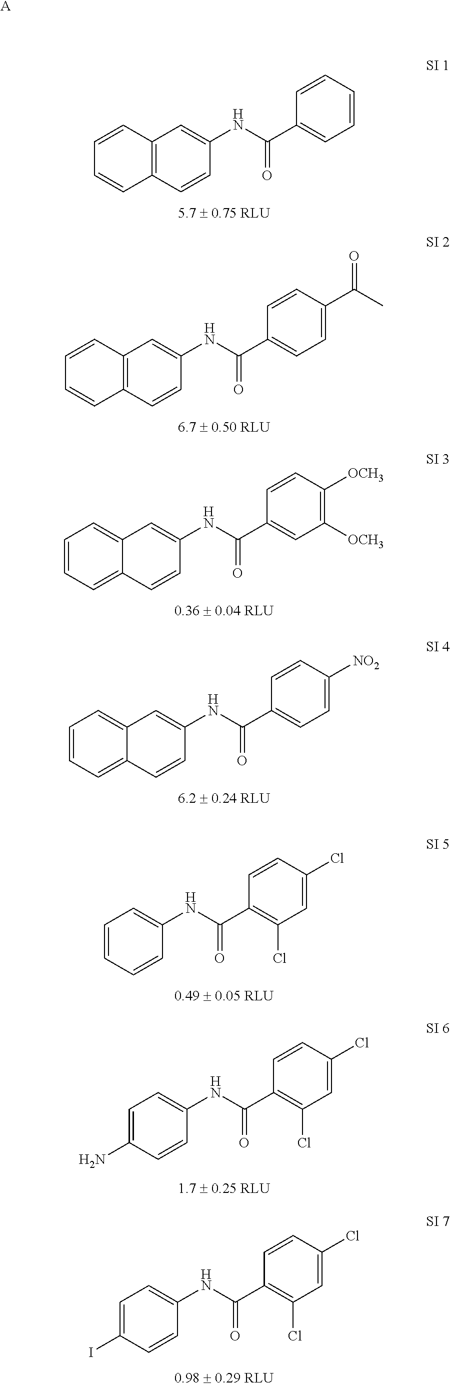

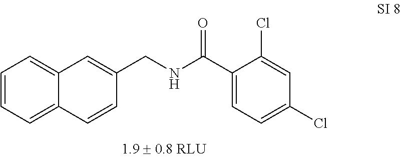 [SMILES: O=C(NCc1ccc2ccccc2c1)c1ccc(Cl)cc1Cl]

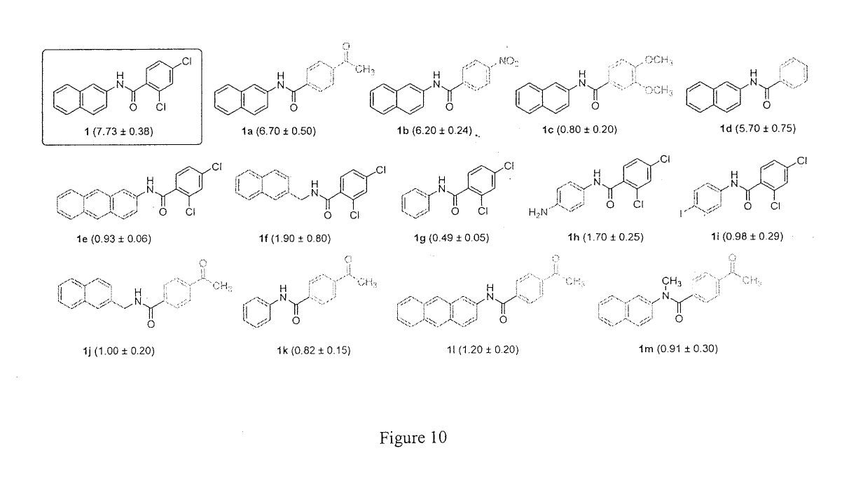

Examples of additional analogs are shown in FIG. 10.

Removal of the halogens, or replacement with an acetyl (1a) or a nitro group (1b) at the 4-position of 1 (7.73±0.38 RLU), resulted in activity changes between 5.7-6.7 RLU. However, replacement of the halogens with methoxy groups at the 3- and 4-position led to a loss of activity. Modification of the naphtyl group was much less tolerated, since replacement with a larger anthracenyl (1e) or a methylene naphtyl group (1f) led to virtually complete loss of miR-122 inhibitory activity. Replacement of the napthyl group with a phenyl (1g), p-aminophenyl (1h) or p-iodophenyl (1i), group led to a complete loss of miR-122 inhibitory activity. Similar changes of the naphtyl ring in the miR-122 inhibitor 1a also led to complete loss of activity (1j-1l). The insertion of a methylene unit between the amide nitrogen and the napthyl group also led to a substantial loss of activity (1m).

The structure-activity relationship of the second, more potent miR-122 inhibitor 2 (12.51±1.25 RLU) was investigated. The following compounds are analogs of miR-122 inhibitor 2.

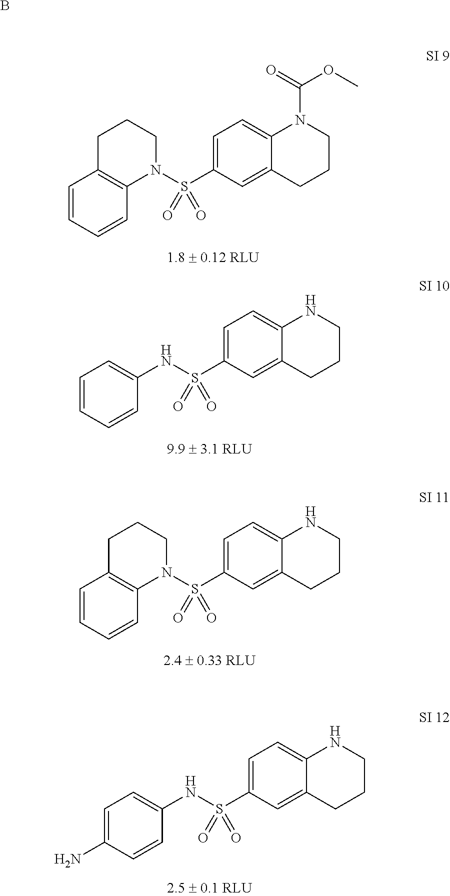

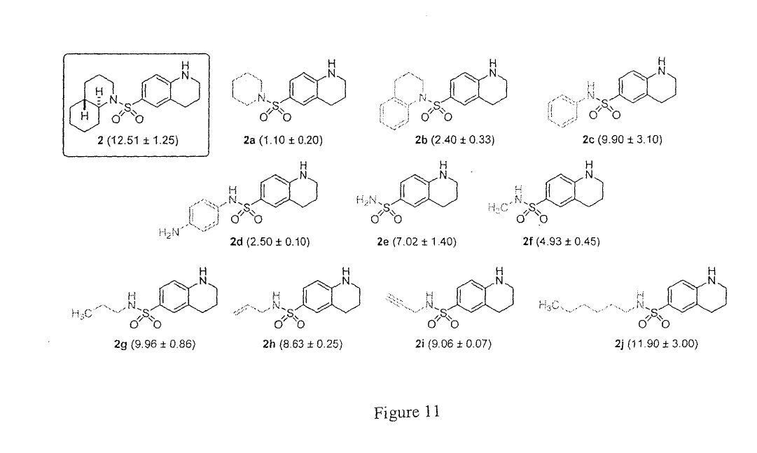

Examples of additional analogs are shown in FIG. 11.

Structural modifications of the miR-122 activator 3 displayed a retention of the activity upon removal of the NH₂ group (0.18±0.06 RLU), but a reduction in activity after removal of both methyl groups from the tertiary amine (0.39±0.16 RLU). However, activity was completely lost upon the installation of a sterically demanding napthyl imide substituent, or introduction of aromatic nitro groups. Activity was lost upon acylation of the NH group; however, an only slightly diminished activity (9.9±3.1 RLU) was observed when the sulfonamide was connected to aniline, as opposed to the decahydroquinoline. Replacing the decahydroquinoline with piperidine (2a) or dihydroquinoline (2b) led to a loss of activity. Activity was maintained in the case of aniline (2c) but was lost through installation of a p-amino group (2d). The simple sulfonamide 2e showed a 50% reduced activity which was then gradually improved almost to the level of the parent compound 2 by installing carbon chains of increasing length, from methyl (20, propyl (2g), allyl (2h) and propargyl (2i), to hexyl (2j). The acylation of the NH group of any of the miR-122 inhibitors shown in FIG. 11 with CO₂CH₂CH₃ led to a complete abrogation of activity.

Some analogs of miR-122 activator 3 are:

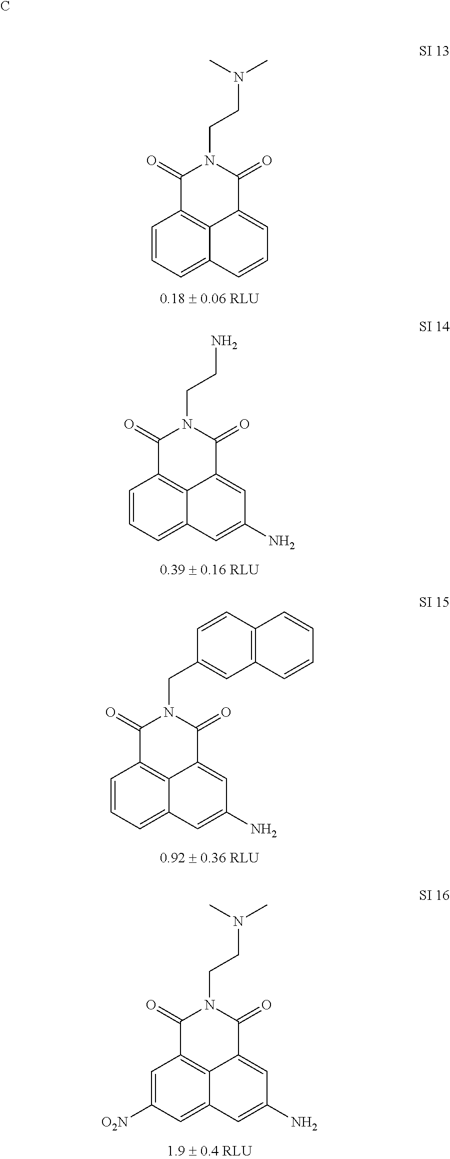

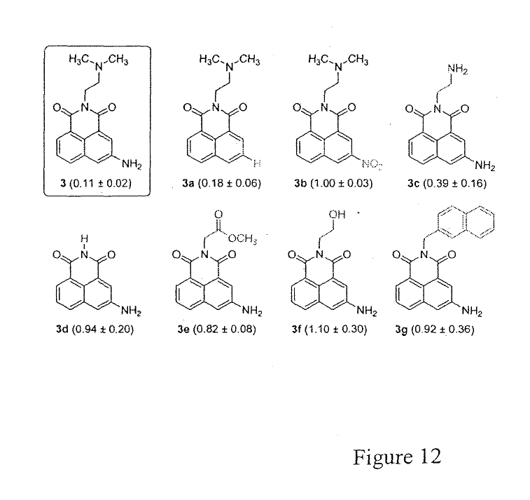

Examples of additional analogs are shown in FIG. 12.

Removal of the NH₂ group (3a) displayed retention of activity; however, replacement with a nitro group (3b) completely abrogated miR-122 activation. The removal of both N-methyl groups (3c) and substitution of the ethylamino group with a proton (3d) led to an increasing loss of activity. Other modifications of that group, e.g. with a methylcarboxylate (3e), ethylhydroxy (3f), or a naphtyl group (3g), also induced a complete loss of miR-122 activation.

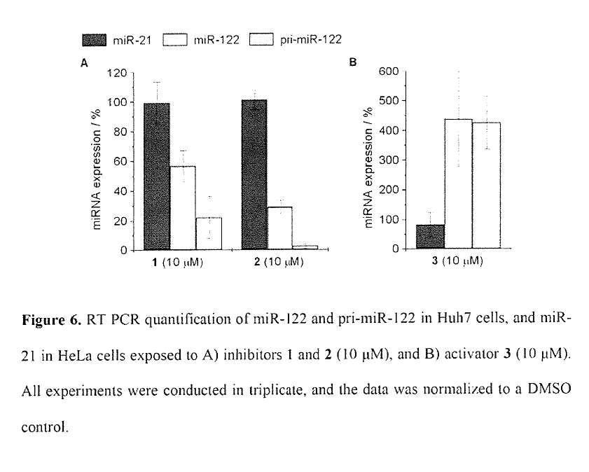

The activity of the small molecule modifiers was analyzed by quantitative RT PCR in order to measure their direct effects on miR-122 expression levels in Huh7 cells. Cells were incubated with compounds 1-3 (10 μM) for 48 hours, followed by total RNA isolation (miR Premier miRNA Isolation Kit; Aldrich), and quantification by RT PCR using TaqMan primers (Applied Biosystems). The sulfonamide 2 elicited a 72% knock-down of mature miR-122 levels relative to DMSO treated cells (FIG. 6). FIG. 6 shows RT PCR quantification of miR-122 and pri-miR-122 in Huh7 cells, and miR-21 in HeLa cells exposed to A) inhibitors 1 and 2 (10 μM), and B) activator 3 (10 μM). All experiments were conducted in triplicate, and the data was normalized to a DMSO control. The amide 1 displayed only a 45% knock-down, which is consistent with the lower activity of compound 1 observed in the functional assay. The small molecule activator 3 led to a 438% increase in miR-122 expression levels. In case of the inhibitors a significant reduction in copy number was also observed by quantitative RT PCR with primers specifically designed to be unique to the pri-miR-122 sequence (FIG. 6),¹⁴ indicating a down-regulation to 22% and 3% for compound 1 and 2, respectively. Thus, the miR-122 inhibitors 1 and 2 seem to be targeting the transcription of the miRNA gene into primary miRNA, rather than other components of the miRNA pathway. The pri-miR-122 levels are reduced further than the miR-122 levels in the presence of the small molecule inhibitors. All compounds were also additionally assessed by RT PCR for miR-21, and within the error margin, no effect was observed. This confirms the data obtained in the luciferase assays and again suggests a degree of specificity of the small molecule modifiers 1-3 for miR-122.

The present invention includes all possible pharmaceutically acceptable salts, solvates, stereoisomers, tautomers and geometric isomers of the compounds of structures I, II and III and compounds 1, 2, and 3 and their analogs and includes not only racemic compounds but also the optically active isomers as well. When a compound is desired as a single enantiomer, it may be obtained either by resolution of the final product or by stereospecific synthesis from either isomerically pure starting material or any convenient intermediate. Resolution of the final product, an intermediate or a starting material may be effected by any suitable method known in the art for example Chiral reagents for Asymmetric Synthesis by Leo A. Paquette; John Wiley & Sons Ltd. Additionally, in situations wherein tautomers of the compounds are possible, the present invention is intended to include all tautomeric forms of the compounds.

Certain compounds can exist in solvated forms including hydrated forms.

The present invention also includes within its scope all isotopically labeled forms of compounds that activate, inhibit or modulate miR-122 wherein one or more atoms of the compounds is replaced by their respective isotopes. Examples of isotopes that may be incorporated into the compounds disclosed herein include, but are not limited to, isotopes of hydrogen such as ²H and ³H, carbon such as ¹¹C, ¹³C and ¹⁴C, nitrogen such as ¹³N and ¹⁵N, oxygen such as ¹⁵O, ¹⁷O and ¹⁸O, chlorine such as ³⁶Cl, fluorine such as ¹⁸F and sulphur such as ³⁵S.

Substitution with heavier isotopes, for example, replacing one or more key carbon-hydrogen bonds with carbon-deuterium bond may show certain therapeutic advantages, for example, longer metabolism cycles, improved safety or greater effectiveness.

Isotopically labeled forms of compounds can be prepared by conventional techniques known to those skilled in the art or by processes analogous to those described in the subsequent experimental section by using an appropriate isotopically labeled reagent instead of non-labeled reagent.

Pharmaceutically acceptable salts may be synthesized from the compounds by conventional chemical methods. Generally, such salts may be prepared by reacting the free acid or base forms of a compound with a stoichiometric amount of the appropriate base or acid in water or in an organic solvent, or in a mixture of the two. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts may be synthesized from the parent compound which contains a basic or acidic moiety by conventional chemical methods. Generally, such salts may be prepared by reacting the free acid or base forms of these compounds with a stoichiometric amount of the appropriate base or acid in water or in an organic solvent, or in a mixture of the two.

According to another aspect of the present invention, there is provided a pharmaceutical composition comprising a compound as described above as an active ingredient, or a pharmaceutical salt thereof, in association with a pharmaceutically acceptable carrier or excipient.

Pharmaceutical compositions of the invention can also include conventional pharmaceutical carriers, excipients and/or additives. Suitable pharmaceutical carriers, excipients and additives include stabilizers, antioxidants, osmolality adjusting agents, buffers, pH adjusting agents, fillers, dispersants, emulsifiers, defoamers, sweeteners, flavors, preservatives, solubilizers and colorants. A composition can also contain one or more additional therapeutically or prophylactically active compounds and/or their pharmaceutically acceptable salts.

Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms are dictated by and directly dependent on (a) the unique characteristics of the therapeutic compound and the particular therapeutic effect to be achieved, and (b) the limitations inherent in the art of compounding.

The pharmaceutical compositions featured in the invention may be administered in a number of ways depending upon whether local or systemic treatment is desired and upon the area to be treated. Administration may be topical (including ophthalmic, intranasal, transdermal, intrapulmonary), mucosal, pulmonary, oral or parenteral. Parenteral administration includes intravenous drip, subcutaneous, intraperitoneal or intramuscular injection, or intrathecal or intraventricular administration.

The compositions may be in a form suitable for i) oral use, for example, aqueous or oily suspensions, dispersible powders or granules, elixirs, emulsions, hard or soft capsules, lozenges, syrups, tablets or trouches; ii) topical use, for example, creams, ointments, transdermal patches, gels, aqueous or oily solutions or suspensions, or iii) parenteral administration, for example, sterile aqueous or oily solution for intravenous, subcutaneous, intraperitoneal, intramuscular or as a suppository for rectal dosing.

The pharmaceutical preparations according to the invention are prepared in a manner known per se and familiar to one skilled in the art.

Actual dosage levels of the active ingredients in the pharmaceutical compositions of this invention may be varied so as to obtain an amount of the active ingredient, which is effective to achieve the desired therapeutic response for a particular patient, composition, and mode of administration without causing undue side effects or being toxic to the patient.

One skilled in the art will appreciate that the exact individual dosages may be adjusted somewhat depending on a variety of factors, including the specific compound being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns are preferably determined by the attending physician in consideration of the above-identified factors.

A compound or composition may be administered either simultaneously or before or after a second active ingredient, either separately by the same or different route of administration or together in the same pharmaceutical formulation.

In certain methods of the invention, there is a further step of administering the selected miRNA modulator, activator or inhibitor to a cell, tissue, organ, or organism (collectively “biological matter”) in need of treatment related to modulation of the targeted miRNA or in need of the physiological or biological results discussed herein.

The compounds and compositions of this invention or those identified through the assay described herein can be used in the prevention and/or treatment of diseases and conditions of the liver or other organ where miR-122 is present. These compounds and compositions can be used in the prevention or treatment of liver cancer, or the prevention or treatment of HCV infection, chronic hepatitis or cirrhosis.

Furthermore, it is contemplated that the miRNA compositions may be provided as part of a therapy to a patient, in conjunction with traditional therapies or preventative agents. Moreover, it is contemplated that any method discussed in the context of therapy may be applied preventatively, particularly in a patient identified to be potentially in need of the therapy or at risk of the condition or disease for which a therapy is needed.

The compounds of this invention are useful as tools in the development and standardization of in vitro and in vivo tests for the evaluation of the effects on miR-122, prevention or treatment of HCC and/or treatment of HCV.

The present invention also concerns kits containing compositions or compounds described herein, compounds or compositions to implement methods of the invention. In some embodiments, kits can be used to evaluate one or more molecules as miRNA inhibitors or activators.

Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2, 5×, 10×, or 20× or more.

It is to be understood that other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances.

All publications and patent applications in this specification are indicative of the level of ordinary skill in the art to which this invention pertains.

The invention has been described with reference to various specific and preferred embodiments and techniques and is not limited in scope by the exemplified embodiments, which are intended as illustrations of single aspects of the invention only. However, it should be understood that many variations and modifications may be made while remaining within the spirit and scope of the invention.

The invention is further understood by reference to the following examples, which are intended to be purely exemplary of the invention. Various modifications of the invention in addition to those described herein will become apparent to those skilled in the art from the foregoing description. Such modifications fall within the scope of the appended claims.

### EXAMPLES

### Example 1

### Reporter Plasmid Construction

The psiCHECK-2 plasmid (1 μg; Promega) was sequentially digested with SgfI (10 units, 50 μL reaction; Promega) followed by PmeI (10 units; New England Biolabs) and gel purified. Insert DNA containing the miR122 binding site was purchased from IDT DNA (5′ CGCAGTAGAGCTCTAGTACAAACACCATTGTCACACTCCAGTTT 3′ (SEQ ID NO:1) and 5′ AACTGGAGTGTGACAATGGTGTTTGTACTAGAGCTCTA CTGCGAT 3′) (SEQ ID NO:2) and hybridized (90° C., cooled to 4° C. over 5 min, then 4° C. for 60 min) and ligated with T4 ligase (200 units, 10 μL reaction, 1:10 vector/insert ratio; New England Biolabs) into the digested psiCHECK-2 vector. Positive colonies were selected by PCR colony screens and the construction of the psiCHECK-miR122 vector was confirmed by sequencing (sequencing primer: 5′ GCTAAGAAGTTCCCT 3′ (SEQ ID NO:3); IDT DNA).

### Example 2

### Assessment of psiCHECK-miR122Reporter System

Experiments were performed using Huh7, HeLa, and HeLa-miR-21-Luc cell lines cultured in Dulbecco's Modified Eagle Medium (DMEM; Hyclone) supplemented with 10% Fetal Bovine Serum (FBS; Hyclone) and 2% streptomycin/ampicillin (MP Biomedicals) and maintained at 37° C. in a 5% CO₂ atmosphere. Hep2G cells were cultured in Eagle's Minimum Essential Medium (Hyclone) supplemented with 10% FBS (Hyclone) and 0.5 mM sodium pyruvate.

Huh7 and HeLa cells were transfected at approximately 60% confluency with either the psiCHECK-Control plasmid (the original psiCHECK-2 plasmid containing no known miRNA binding site) or the psiCHECK-miR122 plasmid (0.5 μg) using X-tremGENE transfection reagent (3:2 reagent/DNA ratio; Roche) in Opti-Mem media (Invitrogen). All transfections were performed in triplicate for statistical analysis. The cells were incubated at 37° C. for 4 hours followed by the replacement of transfection media with standard DMEM growth media. After 48 hours of incubation the media was removed, cells were lysed and assayed with a Dual Luciferase Assay Kit (Promega) using a Wallac VICTOR³V luminometer with a measurement time of 1 s and a delay time of 2 s. The ratio of Renilla to firefly luciferase expression was calculated for each of the triplicates, the data was averaged, and standard deviations were calculated (FIG. 2).

### Example 3

### Assay for Small Molecule Effectors of miR-122

Huh7 cells were transfected with the psiCHECK-miR122 plasmid in 96-well plates as previously described. After 4 hours of incubation the transfection media was removed and replaced with DMEM growth media (100 μL) supplemented with 10 μM of the small molecules (NCI Diversity Set II; 1% DMSO final concentration). Cells were incubated for 48 hours followed by analysis with a Dual Luciferase Assay Kit (Promega) as previously described.

### Example 4

### Validation of miR-122 Small Molecule Inhibitors

Compounds which elicited greater than a 5-fold increase of the relative luciferase units signal compared to the DMSO control were then re-assayed in triplicate to confirm the validity of the hit compound. Upon secondary validation of miR-122 inhibition, identical assays were repeated with the psiCHECK-Control vector to ascertain that the effect is specific to the miR-122 pathway, and not simply affecting the Renilla gene. In order to assess the relative degree of miR-122 inhibition, Huh7 cells were also co-transfected with either the psiCHECK-Control plasmid or the psiCHECK-miR122 plasmid (0.5 μg) and a miR-122 2′-OMe PS antagomir (50 pmol; 5′ ACAAACACCAUUGUCACACUCCA (SEQ ID NO:4) 3′; IDT DNA)(2) using the X-tremGENE transfection reagent (Roche Applied Science, 4:2 reagent/DNA ratio). The transfection media was replaced with standard DMEM growth media after 4 hours and the cells were incubated for 48 hours prior to analysis with a Dual Luciferase Assay Kit (FIG. 3).

### Example 5

### Examination of Small Molecule Inhibitor Specificity for miR-122

Using the previously described HeLa-miR-21-Luc cell line (a stably transfected line harboring a miR-21 binding sequence in the 3′ UTR of a firefly luciferase gene),(9) cells were grown to 80% confluency (DMEM) and treated with either DMSO, the previously reported miR-21 inhibitor, or compounds 1 and 2 at 10 μM (1% DMSO final concentration). The cells were incubated for 48 hours at 37° C. (5% CO₂) and then assayed with a Bright-Glo Luciferase Kit (Promega) and analyzed using a Wallac VICTOR³V luminometer with a measurement time of 1 s and a delay time of 2 s (FIG. 4).

### Example 6

### Quantitative Real Time PCR Analysis

Huh7 cells were passaged into 6-well plates, grown to 60% confluency, and treated with compounds 1-2 (10 μM) or DMSO (10% final DMSO concentration). Each treatment was conducted in triplicate to ensure statistical validity. Cells were then incubated at 37° C. for 48 hours (DMEM, 5% CO₂). The media was removed, and cells were washed with PBS buffer (2×2 mL, pH 7.4) followed by RNA isolation with the mirPremier™ microRNA Isolation Kit (Aldrich). The RNA was quantified using a Nanodrop ND-1000 spectrophotometer and 10 ng of each RNA sample was reverse transcribed using the TaqMan™ microRNA Reverse Transcription Kit (Applied Biosystems) in conjunction with either the miR-122 or miR-21 TaqMan™ RT primer (16° C., 30 min; 42° C., 30 min; 85° C., 5 min). Quantitative Real Time PCR was conducted with a TaqMan™ 2× Universal PCR Master Mix and the appropriate TaqMan™ miRNA assay (Applied Biosystems) on a BioRad MyiQ RT-PCR thermocycler (1.3 μL RT PCR product; 95° C., 10 min; followed by 40 cycles of 95° C., 15 s; 60° C. 60 s). Threshold cycles were used to determine miRNA copy numbers, and the levels of miR-122 and miR-21 were compared via determination of threshold cycle (Cₜ) and conversion to copy number (copy number=10[(Ct−37.4)/−3.3]).¹⁷ The data was then normalized to the DMSO control. The samples were also analyzed by real time PCR for the presence of pri-miR-122 transcript using the previously described primers (5′ GCTCTTCCCATTGCTCAAGATG 3′ (SEQ ID NO:5) and 5′ GTATGTAACAACAGCATGTG 3′ (SEQ ID NO:6); IDT DNA) and iQ SYBR Green Supermix for the real time PCR (95° C., 3 min; followed by 40 cycles of 95° C., 15 s; 60° C. 60 s).¹⁴

### Example 7

### Effect of Small Molecule miR-122 Inhibitors on HCV Replication

The pHtat2Neo/QR/KR/FV/SI plasmid (provided by Dr. Stanley Lemon)¹⁶ was linearized using XbaI (10 units, 50 μL reaction; New England Biolabs), followed by transcription with T7 RNA Polymerase (6 h, 37° C.), and purification on Microcon 10 columns. Huh7 cells were then grown to 60% confluency in a 6 well plate and transfected with 1 μg RNA using X-tremGENE transfection reagent (Roche Applied Science, 3:2 reagent/RNA ratio) in Opti-Mem media (Invitrogen). After a 4 hour incubation at 37° C., the transfection media was removed and replaced with standard growth media (2 mL) supplemented with 10 μM of 1 or 2 (1% DMSO final concentration). All experiments were conducted in triplicate for statistical validation. Cells were incubated for 48 hours at 37° C., followed by media removal and washing with PBS buffer (2×2 mL, pH 7.4), followed by RNA isolation (mirPremier™ microRNA Isolation Kit, Aldrich). Quantitative RT-PCR was then performed as previously described using the general HCV RT-PCR Primers (5′: CGGGAGAGCCATAGTGGTCTGCG 3′ (SEQ ID NO:7) and 5′ CTCGCAAGCACCCTATCAGGCAGTA 3′) (SEQ ID NO:8) and GADPH primers (5′ TGCACCACCAACTGCTTAGC 3′ (SEQ ID NO:9) and 5′ GGCATGGACTGTGGTCATGAG 3′) (SEQ ID NO:10) as a standard control.

### Example 8

### Effects of the miR-122 Activator on Caspase Activity

Both HepG2 and Huh7 cells were passaged into a 96-well plate and grown to 70% confluency. The media was then removed and replaced with standard growth media supplemented with 3 (10 μM) or a DMSO control (1% final DMSO concentration in all wells) and incubated for 48 hours at 37° C. All incubations were conducted in triplicate to ensure statistical validity. The media was removed and cells were assayed with the Caspase-Glo 3/7 (Promega) kit according to the manufacturers directions and luminescence was measured using a Wallac VICTOR³V luminometer with a measurement time of 1 s and a delay time of 2 s.

### Example 9

### Effects of miRNA Small Molecule Modifiers on Cell Viability

HepG2 and Huh7 cells were passaged into a 96-well plate and grown to 60% confluency. The media was then removed and replaced with standard growth media supplemented with increasing concentrations of the miR-122 small molecule modifiers (0-20 μM; 1% final DMSO concentration) and incubated for 48 hours at 37° C. Cellular viability was then assessed using a Cell-Titer Glo Assay (Promega) according to the manufacturers directions and luminescence was measured using a Wallac VICTOR³V luminometer with a measurement time of 1 s and a delay time of 2 s.

### Example 10

### General Protocol for the Synthesis of Analogs SI 1-8

To a solution of the benzoic acid (20 mg) in CH₂Cl₂ (3 mL) was added EDCI (1.5 eq) followed by the amine starting material (1 eq). A catalytic amount of DMAP was then added to the mixture. The reaction mixture was allowed to stir overnight under a nitrogen atmosphere. Water (5 mL) was added and the reaction was extracted with CH₂Cl₂ (3×5 mL). The organic layer was washed with brine, dried over Na₂SO₄, filtered and concentrated. The product was then purified over silica gel eluting with hexane/ethyl acetate to yield the pure products.⁽²⁰⁾

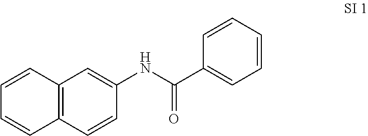 [SMILES: O=C(Nc1ccc2ccccc2c1)c1ccccc1]

### N-(Naphthalen-3-yl)benzamide (SI 1)

¹H NMR (300 MHz, CDCl₃): δ 7.39-7.62 (m, 6H), 7.78-7.86 (m, 3H), 7.90-7.96 (m, 2H), 8.01 (bs, 1H), 8.35 (s, 1H). HRMS Calculated for C₁₇H₁₄NO [M+H]⁺: 248.1075. Found: 248.1048.⁽²¹⁾

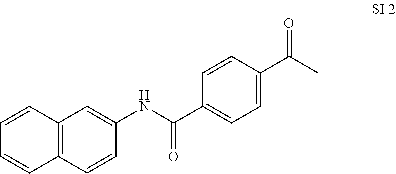 [SMILES: CC(=O)c1ccc(C(=O)Nc2ccc3ccccc3c2)cc1]

### 4-Acetyl-N-(naphthalen-3-yl)benzamide (SI 2)

¹H NMR (300 MHz, CDCl₃): δ 2.67 (s, 3H), 7.40-7.54 (m, 2H), 7.60 (dd, 1H, J=2.1 Hz, J=8.7 Hz), 7.80-7.89 (m, 3H), 8.01 (d, 3H, J=8.1 Hz), 8.09 (d, 2H, J=8.7 Hz), 8.36 (s, 1H). HRMS Calculated for C₁₉H₁₆NO₂[M+H]⁺: 290.1181. Found: 290.1090.

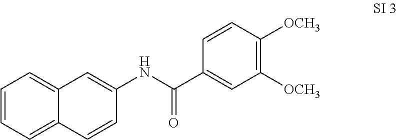 [SMILES: COc1ccc(C(=O)Nc2ccc3ccccc3c2)cc1OC]

### 3,4-dimethoxy-N-(naphthalen-6-yl)benzamide (SI 3)

¹H NMR (300 MHz, CDCl₃): δ 3.93 (s, 6H), 6.88 (d, 1H, J=8.1 Hz), 7.41-7.49 (m, 3H), 7.53 (d, 1H, J=1.8 Hz), 7.61 (dd, 1H, J=2.1 Hz, J=8.7 Hz), 7.80 (t, 3H, J=8.7 Hz), 8.08 (s, 1H), 8.31 (d, 1 H, J=2.1 Hz). HRMS Calculated for C₁₉H₁₆NO₂ [M+H]⁺: 308.1281. Found: 308.2108.

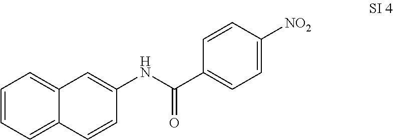 [SMILES: O=C(Nc1ccc2ccccc2c1)c1ccc([N+](=O)[O-])cc1]

### N-(Naphthalen-2-yl)-4-nitrobenzamide (SI 4)

¹H NMR (300 MHz, DMSO): δ 7.43-7.50 (m, 2H), 7.82-7.93 (m, 4H), 8.22 (dd, 2H, J=1.8 Hz, J=6.9 Hz), 8.38 (dd, 2H, J=1.8 Hz, J=6.9 Hz), 8.45 (s, 1H), 10.73 (s, 1H), 10.73 (s, 1H). HRMS Calculated for C₁₉H₁₆NO₂[M+H]⁺: 293.0926. Found: 293.1.

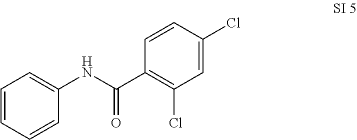 [SMILES: O=C(Nc1ccccc1)c1ccc(Cl)cc1Cl]

### 2,4-Dichloro-N-phenylbenzamide (SI 5)

¹H NMR (300 MHz, CDCl₃): δ 7.18 (t, 1H, J=9 Hz), 7.29-7.43 (m, 4H), 7.63 (t, 3H, J=6 Hz), 8.08 (s, 1H). HRMS Calculated for C₁₃H₁₀Cl₂NO [M+H]⁺: 266.0139. Found: 266.0066.

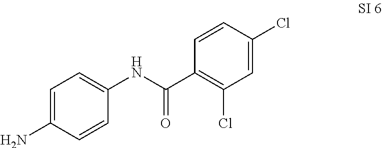 [SMILES: Nc1ccc(NC(=O)c2ccc(Cl)cc2Cl)cc1]

### N-(4-Aminophenyl)-2,4-dichlorobenzamide (SI 6)

¹H NMR (300 MHz, CDCl₃): δ 3.66 (bs, 2H), 6.64-6.70 (m, 2H), 7.30-7.44 (m, 4H), 7.67 (d, 1H, J=8.1 Hz), 7.82 (bs, 1H). HRMS Calculated for C₁₃H₁₁Cl₂N₂O [M+H]⁺: 281.0248. Found: 281.1081.

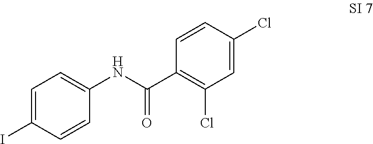 [SMILES: O=C(Nc1ccc(I)cc1)c1ccc(Cl)cc1Cl]

### 2,4-Dichloro-N-(4-iodophenyl)benzamide (SI 7)

¹H NMR (300 MHz, CDCl₃): δ 7.33-7.45 (m, 3H), 7.47 (d, 1H, J=3 Hz), 7.64-7.74 (m, 3H), 7.93 (bs, 1H). HRMS Calculated for C₁₃H₉NOCl₂1 [M+H]⁺: 391.9106. Found: 391.9192.

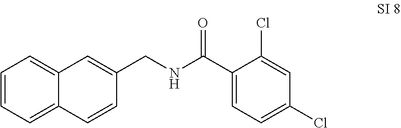 [SMILES: O=C(NCc1ccc2ccccc2c1)c1ccc(Cl)cc1Cl]

### 2,4-Dichloro-N-((naphthalen-6-yl)methyl)benzamide (SI 8)

¹H NMR (300 MHz, CDCl₃): δ 5.09 (d, 2H, J=5.1 Hz), 6.44 (bs, 1H), 7.27 (dd, 1H, J=1.5 Hz, J=8.1 Hz), 7.38 (d, 1H, J=1.8 Hz), 7.42-7.63 (m, 5H), 7.83-7.91 (m, 2H), 8.09 (d, 1H, J=8.1 Hz). HRMS Calculated for C₁₉H₁₆NO₂ [M+H]⁺: 330.0452. Found: 330.0.

### Synthesis of Starting Materials for the Analogs SI 9-12

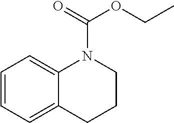

### Methyl 3,4-dihydroquinoline-1(2H)-carboxylate

1,2,3,4-Tetrahydroquinoline (0.412 mL, 3.75 mmol) and K₂CO₃ (3.11 g, 22.52 mmol) were dissolved in acetone (8 mL). CH₃CH₂OCOCl (1.44 mL, 15.02 mmol) was added to the mixture. The reaction mixture was refluxed overnight. The acetone was evaporated and water (5 mL) was added. The reaction was extracted with ethyl acetate (3×5 mL). The organic layer was washed with brine (3×5 mL), dried over Na₂SO₄, filtered, and evaporated to give methyl 3,4-dihydroquinoline-1(2H)-carboxylate in 98% yield. ¹H NMR (300 MHz, CDCl₃): δ 1.32 (t, 3H, J=7.2 Hz), 1.90-1.98 (m, 2H), 2.77 (t, 2H, J=6.3 Hz), 3.76 (t, 2H, J=6.3 Hz), 4.24 (q, 2H, J=7.2 Hz), 6.96-7.18 (m, 3H), 7.67 (d, 1H, J=8.1 Hz). HRMS Calculated for C₁₂H₁₅NO₂ [M+H]⁺: 206.1176. Found: 206.1179.

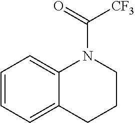 [SMILES: O=C(N1CCCc2ccccc21)C(F)(F)F]

### 2,2,2-Trifluoro-1-(3,4-dihydroquinolin-1(2H)-yl)ethanone

1,2,3,4-Tetrahydroquinoline (0.094 mL, 0.75 mmol) and triethylamine (0.31 mL, 2.25 mmol) were dissolved in Et₂O (1 mL). The mixture was cooled to 0° C. and a solution of (CF₃CO)₂O (0.21 mL, 1.50 mmol) in Et₂O (1 mL) was added dropwise. The reaction mixture was warmed to room temperature and stirred overnight. Water (5 mL) was added and the mixture was extracted with Et₂O (3×5 mL). The organic layer was washed with brine (3×5 mL), dried over Na₂SO₄, filtered, and evaporated to give 2,2,2-trifluoro-1-(3,4-dihydroquinolin-1(2H)-yl)ethanone as an orange/brown solid in 98% yield. ¹H NMR (300 MHz, CDCl₃): δ 2.09 (t, 2H, J=6 Hz), 2.86 (s, 2H), 3.84 (t, 2H, J=6 Hz), 7.19-7.26 (m, 3H), 7.68 (s, 1H). HRMS Calculated for C₁₁H₁₀F₃NO [M+H]⁺: 230.0787. Found: 230.0787.

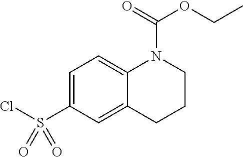 [SMILES: CCOC(=O)N1CCCc2cc(S(=O)(=O)Cl)ccc21]

### Ethyl 6-(chlorosulfonyl)-3,4-dihydroquinoline-1(2H)-carboxylate

Chlorosulfonic acid (0.162 mL, 2.44 mmol) was cooled to 0° C. under a nitrogen atmosphere. Methyl 3,4-dihydroquinoline-1(2H)-carboxylate (0.10 g, 0.49 mmol) was dissolved in CCl₄ (0.5 mL) and was added dropwise to the cooled HSO₃Cl. The solution was allowed to warm to room temperature and was stirred for 2 h. The reaction was quenched with water (5 mL) at 0° C. and was extracted with ethyl acetate (3×5 mL). The organic layer was washed with brine, dried over Na₂SO₄, filtered, and evaporated. The residue was purified over silica gel (8:1 hexane:ethyl acetate) to give the product in 50% yield). ¹H NMR (300 MHz, CDCl₃): δ 1.36 (t, 3H, J=7.2 Hz), 1.96-2.02 (m, 2H), 2.87 (t, 2H, J=6.3 Hz), 3.84 (t, 2H, J=6.3 Hz), 4.30 (q, 2H, J=6.3 Hz), 7.75-7.82 (m, 2H), 8.09 (d, 1H, J=9 Hz).²²

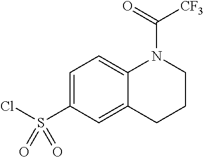 [SMILES: O=C(N1CCCc2cc(S(=O)(=O)Cl)ccc21)C(F)(F)F]

### 1-(2,2,2-Trifluoroacetyl)-1,2,3,4-tetrahydroquinoline-6-sulfonyl chloride

Chlorosulfonic acid (0.043 mL, 0.654 mmol) was cooled to 0° C. under a nitrogen atmosphere. 2,2,2-Trifluoro-1-(3,4-dihydroquinolin-1(2H)-yl)ethanone (30 mg, 0.13 mmol) was dissolved in CCl₄ (0.5 mL) and was added dropwise to the cooled HSO₃Cl. The solution was allowed to warm to room temperature and was stirred for 2 h. The reaction was quenched with water (5 mL) at 0° C. and was extracted with ethyl acetate (3×5 mL). The organic layer was washed with brine, dried over Na₂SO₄, filtered, and evaporated. The residue was purified over silica gel (4:1 hexane:ethyl acetate) to yield the product as a yellow solid (54% yield). ¹H NMR (300 MHz, CDCl₃): δ 2.12-2.20 (m, 2H), 3.02 (t, 2H, J=6.9 Hz), 3.91 (t, 2H, J=6 Hz), 7.87-7.91 (m, 2H), 8.00 (d, 1 H, J=9.6 Hz). HRMS Calculated for C₁H₉ClF₃NO₃S [M−H₂—COCF₃]⁺: 227.9886. Found: 228.0687.

### Synthesis of Analog SI 9

1,2,3,4-Tetrahydroquinoline (0.01 mL, 0.08 mmol) and triethylamine (0.03 mL, 0.20 mmol) were dissolved in CH₂Cl₂ (0.5 mL) and cooled to 0° C. under a nitrogen atmosphere. A solution of ethyl 6-(chlorosulfonyl)-3,4-dihydroquinoline-1(2H)-carboxylate (30 mg, 0.10 mmol) in CH₂Cl₂ (0.5 mL) was then added dropwise to the cooled mixture. The reaction mixture was allowed to warm to room temperature and was stirred overnight. 1 M HCl (3 mL) was added and the reaction was extracted with CH₂Cl₂ (3×5 mL). The organic layer was washed with brine (2×3 mL), dried over Na₂SO₄, filtered, and the volatiles were removed under reduced pressure. The residue was purified by silica gel chromatography (4:1 hexanes:ethyl acetate) to give the pure product ethyl 6-(3,4-dihydroquinolin-1(2H)-ylsulfonyl)-3,4-dihydroquinoline-1(2H)-carboxylate (SI 9) in 45% yield.

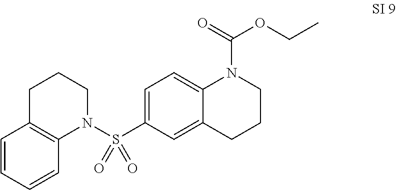 [SMILES: CCOC(=O)N1CCCc2cc(S(=O)(=O)N3CCCc4ccccc43)ccc21]

### Ethyl-6-(3,4-dihydroquinolin-1(2H)-ylsulfonyl)-3,4-dihydroquinoline-1(2H)-carboxylate (SI 9)

¹H NMR (300 MHz, CDCl₃): δ 1.32 (t, 3H, J=7.2 Hz), 1.69-1.71 (m, 2H), 1.89-1.93 (m, 2H), 2.48 (t, 2H, J=6.9 Hz), 2.70 (t, 2H, J=6.6 Hz), 3.74-3.81 (m, 4H), 4.24 (q, 2H, J=6.9 Hz), 7.00-7.08 (m, 2H), 7.16-7.20 (m, 1 μl), 7.33-7.37 (m, 2H), 7.76 (d, 1H, J=8.4 Hz), 7.86 (d, 1H, J=8.4 Hz). HRMS Calculated for C₂₁H₂₄N₂O₄S [M+H]⁺: 401.1530. Found: 401.1529.

### General Protocol for the Synthesis of Analogs SI 10-12

The amine starting material (4 eq) and triethylamine (4 eq) were dissolved in CH₂Cl₂ (0.5 mL) and cooled to 0° C. under a nitrogen atmosphere. A solution of 1-(2,2,2-trifluoroacetyl)-1,2,3,4-tetrahydroquinoline-6-sulfonyl chloride (1 eq) in CH₂Cl₂ (0.5 mL) was then added dropwise to the cooled mixture. The reaction mixture was allowed to warm to room temperature and was stirred overnight. 1 M HCl (3 mL) was added and the reaction was extracted with CH₂Cl₂ (3×5 mL). The organic layer was washed with brine (2×3 mL), dried over Na₂SO₄, filtered, and the volatiles were removed under reduced pressure. The residue was purified by silica gel chromatography (hexanes:ethyl acetate).(7) The TFA protected analogs were directly dissolved in a 10% K₂CO₃ solution in 5:2 CH₃OH/H₂O. The mixture was allowed to stir overnight. The CH₃OH was removed under vacuum and water (3 mL) was added. The reaction mixture was then extracted with CH₂Cl₂ (3×5 mL). The organic layer was washed with water (3×5 mL), dried over Na₂SO₄, filtered, and evaporated to afford the pure products. Yields varied from 26-56% over two steps.

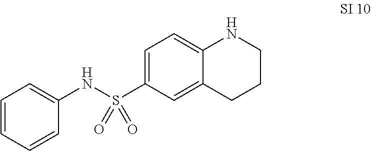 [SMILES: O=S(=O)(Nc1ccccc1)c1ccc2c(c1)CCCN2]

### N-Phenyl-1,2,3,4-tetrahydroquinoline-6-sulfonamide (SI 10)

¹H NMR (300 MHz, CDCl₃): δ 1.84-1.92 (m, 2H), 2.68 (t, 2H, J=6.3 Hz), 3.32 (t, 2H. J=5.7 Hz), 4.32 (bs, 1H), 6.32 (d, 1H, J=9.3 Hz), 6.48 (s, 1H), 7.04-7.09 (m, 3H), 7.21 (d, 2H, J=7.5 Hz), 7.32 (d, 2H, J=6.9 Hz). HRMS Calculated for C₁₉H₁₆NO₂ [M+H]⁺: 289.1011. Found: 289.1.

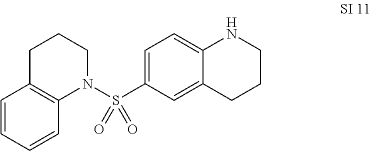 [SMILES: O=S(=O)(c1ccc2c(c1)CCCN2)N1CCCc2ccccc21]

### 6-(3,4-Dihydroquinolin-1(2H)-ylsulfonyl)-1,2,3,4-tetrahydroquinoline (SI 11)

¹H NMR (300 MHz, CDCl₃): δ 1.67-1.72 (m, 2H), 1.87-1.91 (m, 2H), 2.52 (t, 2H, J=6.6 Hz), 2.66 (t, 2H, J=6.3 Hz), 3.33 (t, 2H, J=5.7 Hz), 3.74-3.78 (m, 2H), 4.28 (s, 1H), 6.31 (d, 1H, J=8.1 Hz), 7.02 (q, 2H, J=8.1 Hz), 7.13-7.16 (m, 3H), 7.77 (d, 1 H, J=8.4 Hz). HRMS Calculated for C₁₈H₂₀N₂O₂S [M+Na]⁺: 351.1143. Found: 351.1.

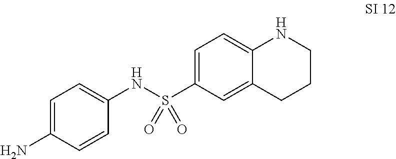 [SMILES: Nc1ccc(NS(=O)(=O)c2ccc3c(c2)CCCN3)cc1]

### N-(4-Aminophenyl)-1,2,3,4-tetrahydroquinoline-6-sulfonamide (SI 12)

¹H NMR (300 MHz, CDCl₃): δ 1.87-1.92 (m, 2H), 2.69 (t, 2H, J=6 Hz), 3.33 (t, 2H, J=5.7 Hz), 6.03 (s, 1H), 6.31 (d, 1H, J=8.7 Hz), 6.54 (d, 2H, J=8.7 Hz), 6.84 (d, 2H, J=8.7 Hz), 7.19 (d, 1H, J=2.4 Hz). HRMS Calculated for C₁₅H₁₈N₃O₂S [M+H]⁺: 304.112. Found: 304.1123.

### Synthesis of Analogs SI 13-16

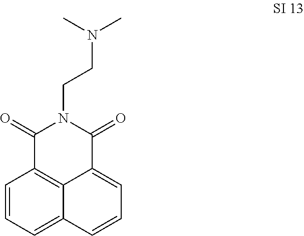 [SMILES: CN(C)CCN1C(=O)c2cccc3cccc(c23)C1=O]

### 2-(2-(Dimethylamino)ethyl)-1H-benzo[de]isoquinoline-1,3(2H)-dione (SI 13)

1,8-Naphthalic anhydride (50 mg, 0.252 mmol) was suspended in ethanol (3 mL) under a nitrogen atmosphere. The mixture was heated to 65° C. and N,N-dimethylethylenediamine (0.029 mL, 0.265 mmol) was added. The reaction mixture was allowed to reflux for 2 h, and was subsequently cooled to 0° C. in an ice bath. A precipitate formed, was collected by filtration, and was washed with ethanol (2×5 mL) and hexanes (5 mL). The solid was dried under vacuum to give the pure product SI 13 as a tan solid (65 mg, 96% yield). ¹H NMR (300 MHz, CDCl₃): δ 2.19 (s, 6H), 2.49 (t, 2H, J=6.9 Hz), 4.14 (t, 2H, J=6.9 Hz), 7.86 (t, 2H, J=7.2 Hz), 8.43-8.49 (m, 4H). HRMS Calculated for C₁₉H₁₆NO₂[M+H]⁺: 269.129. Found: 269.1.²⁴

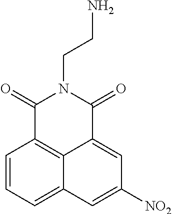 [SMILES: NCCN1C(=O)c2cccc3cc([N+](=O)[O-])cc(c23)C1=O]

### 2-(2-Aminoethyl)-5-nitro-1H-benzo[de]isoquinoline-1,3(2H)-dione

3-Nitro-1,8-naphthalic anhydride (50 mg, 0.206 mmol) was suspended in ethanol (2 mL) under a nitrogen atmosphere. The mixture was heated to 65° C. and ethylenediamine (0.055 mL, 0.822 mmol) was added. The reaction mixture was heated to reflux for 2 h. The reaction mixture was cooled to 0° C. in an ice bath, and the precipitate was collected by filtration and was washed with ethanol (2×5 mL) and hexanes (5 mL). The solid was dried under vacuum to give the pure product (50 mg, 85% yield). ¹H NMR (300 MHz, CDCl₃): δ 3.10 (t, 2H, J=6.6 Hz), 4.31 (t, 2H, J=6.6 Hz), 7.94 (t, 1H, J=7.2 Hz), 8.42 (d, 1H, J=8.7 Hz), 8.79 (d, 1H, J=8.7 Hz), 9.12 (s, 1H), 9.31 (s, 1H). HRMS Calculated for C₁₄H₁₁N₃O₄[M+H]⁺: 286.0822. Found: 286.0824.

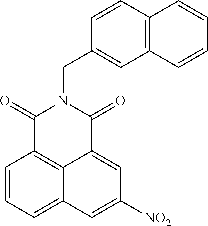 [SMILES: O=C1c2cccc3cc([N+](=O)[O-])cc(c23)C(=O)N1Cc1ccc2ccccc2c1]

### 2-(Naphthalen-2-ylmethyl)-5-nitro-1H-benzo[de]isoquinoline-1,3(2H)-dione

3-Nitro-1,8-naphthalic anhydride (50 mg, 0.206 mmol) was suspended in ethanol (3 mL) under a nitrogen atmosphere. The mixture was heated to 65° C. and 2-naphthalenemethylamine (0.031 mL, 0.216 mmol) was added. The reaction mixture was heated to reflux for 2 h. The reaction mixture was cooled to 0° C. in an ice bath, and the precipitate was collected by filtration and was washed with ethanol (2×5 mL) and hexanes (5 mL). The solid was dried under vacuum to give the pure product (75.5 mg, 96% yield). ¹H NMR (300 MHz, CDCl₃): δ 5.92 (s, 2H), 7.33-7.42 (m, 2H), 7.52 (t, 1H, J=6.9 Hz), 7.62 (t, 1H, J=6.9 Hz), 7.77 (d, 1H, J=7.8 Hz), 7.88 (d, 1H, J=8.1 Hz), 7.96 (t, 1H, J=7.5 Hz), 8.35 (d, 1H, J=8.4 Hz), 8.45 (d, 1H, J=7.8 Hz), 8.82 (d, 1H, J=7.5 Hz), 9.15 (s, 1H), 9.36 (s, 1H). HRMS Calculated for C₂₃H₁₄N₂O₄ [M+Na]⁺: 405.0846. Found: 405.0849.

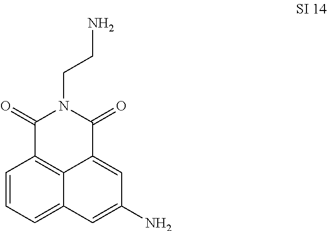 [SMILES: NCCN1C(=O)c2cccc3cc(N)cc(c23)C1=O]

### 5-Amino-2-(2-aminoethyl)-1H-benzo[de]isoquinoline-1,3(2H)-dione (SI 14)

2-(2-Aminoethyl)-5-nitro-1H-benzo[de] isoquinoline-1,3(2H)-dione (10 mg, 0.035 mmol) was dissolved in 4 mL of THF/EtOH (1:1) under a nitrogen atmosphere. 10% Pd/C (3 mg) was added to the solution and the mixture was stirred overnight under a hydrogen atmosphere (H₂ balloon). The reaction mixture was filtered over celite and concentrated in vacuo to give the pure product SI 14 in 56%. ¹H NMR (300 MHz, CDCl₃): δ 2.98 (t, 2H, J=6.6 Hz), 4.13-4.21 (m, 4H), 7.20 (s, 1H), 7.53 (t, 1H, J=7.8 Hz), 7.86 (d, 1H, J=8.4 Hz), 7.94 (s, 1H), 8.24 (d, 1H, J=7.5 Hz). HRMS Calculated for C₁₄H₁₄N₃O₂ [M+H]⁺: 256.1086. Found: 256.1089.

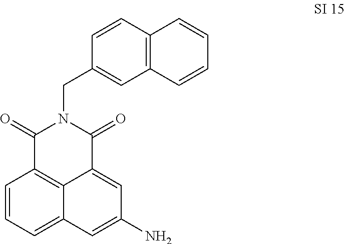 [SMILES: Nc1cc2c3c(cccc3c1)C(=O)N(Cc1ccc3ccccc3c1)C2=O]

### 5-amino-2-(naphthalen-2-ylmethyl)-1H-benzo[de] isoquinoline-1,3(2H)-dione (SI 15)

2-(Naphthalen-2-ylmethyl)-5-nitro-1H-benzo[de] isoquinoline-1,3(2H)-dione (10 mg, 0.026 mmol) was dissolved in 4 mL of THF/EtOH (1:1) under a nitrogen atmosphere. 10% Pd/C (3 mg) was added to the solution and the mixture was stirred overnight under a hydrogen atmosphere (H₂ balloon). The reaction mixture was filtered over celite and concentrated in vacuo to give the pure product SI 15 in 27% yield. ¹H NMR (300 MHz, CDCl₃): δ 4.14 (s, 2H), 5.88 (s, 2H), 7.32-7.35 (m, 3H), 7.52 (t, 1 H, J=8.1 Hz), 7.59 (q, 2H, J=8.1 Hz), 7.73-7.75 (m, 1H), 7.86 (d, 1H, J=7.5 Hz), 7.96 (d, 1H, J=7.8 Hz), 8.06 (s, 1H), 8.34 (t, 2H, J=8.1 Hz). HRMS Calculated for C₂₃H₁₇N₂O₂ [M+H]⁺: 353.129. Found: 353.1289.

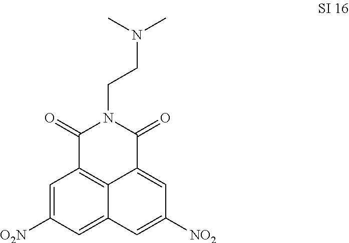 [SMILES: CN(C)CCN1C(=O)c2cc([N+](=O)[O-])cc3cc([N+](=O)[O-])cc(c23)C1=O]

### 2-(2-(Dimethylamino)ethyl)-5,8-dinitro-1H-benzo[de] isoquinoline-1,3(2H)-dione (SI 16)

1,8-Naphthalic anhydride (0.100 g, 0.5 mmol) was dissolved in concentrated H₂SO₄ (1 mL) and was cooled to 0° C. HNO₃ (0.1 mL) was added dropwise to the solution, the reaction was allowed to warm to room temperature, and was then heated to 60° C. for 3 h. Water (2 mL) was added to precipitate the product. The precipitate was filtered and washed with glacial acetic acid (2 mL), followed by toluene (2 mL). The product was then dried under vacuum (34% yield). The dinitro anhydride (0.050 g, 0.174 mmol) was suspended in ethanol (2 mL) under nitrogen. The mixture was heated to 65° C., N,N-dimethylethylenediamine (0.02 mL, 0.183 mmol) was added, and the reaction mixture was heated to reflux for 2 h. The reaction mixture was cooled to 0° C. in an ice bath, and the precipitate was collected by filtration. The precipitate was washed with ethanol (2×5 mL) and hexanes (5 mL). The solid was dried under vacuum to give the pure product SI 16 (25 mg) in 45% yield. ¹H NMR (300 MHz, DMSO): δ 2.28 (s, 6H), 2.65 (t, 2H), 4.21 (t, 2H, J=6.6 Hz), 9.06 (d, 2H, J=2.4 Hz), 9.74 (d, 2H, J=2.1 Hz). HRMS Calculated for C₁₉H₁₆NO₂ [M+H]⁺: 359.0992. Found: 359.1001.²⁵

### REFERENCES

1. (a) Pillai, R. S.; Bhattacharyya, S. N.; Filipowicz, W., Trends Cell Biol 2007, 17, 118-126. (b) Chan, S. P.; Slack, F. J., RNA Biol 2006, 3, 97. (b) Filipowicz, W., Cell 2005, 122, 17-20.
2. Esquela-Kerscher, A.; Slack, F. J., Nat Rev Cancer 2006, 6, 259.
3. Chang, J.; Nicolas, E.; Marks, D.; Sander, C.; Lerro, A.; Buendia, M. A.; Xu, C.; Mason, W. S.; Moloshok, T.; Bort, R.; Zaret, K. S.; Taylor, J. M., RNA Biol 2004, 1, 106-113
4. (a) Formari, F.; Gramantieri, L.; Ferracin, M.; Veronese, A.; Sabbioni, S.; Calin, G. A.; Grazi, G. L.; Giovannini, C.; Croce, C. M.; Bolondi, L.; Negrini, M., Oncogene 2008, 27, 5651-5661. (b) Gramantieri, L.; Ferracin, M.; Formari, F.; Veronese, A.; Sabbioni, S.; Liu, C. G.; Calin, G. A.; Giovannini, C.; Ferrazzi, E.; Grazi, G. L.; Croce, C. M.; Bolondi, L.; Negrini, M., Cancer Res 2007, 67, 6092-6099.
5. Parkin, D. M.; Bray, F.; Ferlay, J.; Pisani, P., CA Cancer J Clin 2005, 55, 74-108.
6. (a) Jopling, C. L.; Yi, M.; Lancaster, A. M.; Lemon, S. M.; Sarnow, P., Science 2005, 309, 1577-1581. (b) Jopling, C. L.; Norman, K. L.; Sarnow, P., Cold Spring Harb Symp Quant Biol 2006, 71, 369-376.
7. Esau, C.; Davis, S.; Murray, S. F.; Yu, X. X.; Pandey, S. K.; Pear, M.; Watts, L.; Booten, S. L.; Graham, M.; McKay, R.; Subramaniam, A.; Propp, S.; Lollo, B. A.; Freier, S.; Bennett, C. F.; Bhanot, S.; Monia, B. P., Cell Metab 2006, 3, 87-98.
8. Elmen, J.; Lindow, M.; Schutz, S.; Lawrence, M.; Petri, A.; Obad, S.; Lindholm, M.; Hedtjarn, M.; Hansen, H. F.; Berger, U.; Gullans, S.; Kearney, P.; Sarnow, P.; Straarup, E. M.; Kauppinen, S., Nature 2008, 452, 896-899.
9. Gumireddy, K.; Young, D. D.; Xiong, X.; Hogenesch, J. B.; Huang, Q.; Deiters, A., Angew Chem Int Ed 2008, 47, 7482-7484.
10. Shan, G.; Li, Y.; Zhang, J.; Li, W.; Szulwach, K. E.; Duan, R.; Faghihi, M. A.; Khalil, A. M.; Lu, L.; Paroo, Z.; Chan, A. W.; Shi, Z.; Liu, Q.; Wahlestedt, C.; He, C.; Jin, P., Nat Biotechnol 2008, 26, 933-940.
11. Cheng, A. M.; Byrom, M. W.; Shelton, J.; Ford, L. P., Nucleic Acids Res 2005, 33, 1290-1297.
12. Zhang, J. H.; Chung, T. D.; Oldenburg, K. R., J Biomol Screen 1999, 4, 67-73.
13. Lin, C. J.; Gong, H. Y.; Tseng, H. C.; Wang, W. L.; Wu, J. L., Biochem Biophys Res Commun 2008, 375, 315.
14. Tsai, W. C.; Hsu, P. W.; Lai, T. C.; Chau, G. Y.; Lin, C. W.; Chen, C. M.; Lin, C. D.; Liao, Y. L.; Wang, J. L.; Chau, Y. P.; Hsu, M. T.; Hsiao, M.; Huang, H. D.; Tsou, A. P., Hepatology 2009, 49, 1571-1582.
15. Pedersen, I. M.; Cheng, G.; Wieland, S.; Volinia, S.; Croce, C. M.; Chisari, F. V.; David, M., Nature 2007, 449, 919-922.
16. Yi, M.; Lemon, S. M., J Virol 2004, 78, 7904-7915.
17. Medhurst, A. D.; Pangalos, M. N., Methods Mol Med 2003, 79, 229-241.
18. Gatfield, D., et al. (2009) Integration of microRNA miR-122 in hepatic circadian gene expression. Genes Dev 23:1313-1326.
19. Krutzfeldt, J., et al. (2005) Silencing of microRNAs in vivo with ‘antagomirs’. Nature 438:685-689.
20. Tanaka, F., Mase, N., Barbas, C. F., 3rd (2004) Design and use of fluorogenic aldehydes for monitoring the progress of aldehyde transformations. J Am Chem Soc 126:3692-3693.
21. Chen, C. T., et al. (2005) Nucleophilic acyl substitutions of anhydrides with protic nucleophiles catalyzed by amphoteric, oxomolybdenum species. J Org Chem 70:1188-1197.
22. Letellier, M. A., et al. (2008) Synthesis of potential Rho-kinase inhibitors based on the chemistry of an original heterocycle: 4,4-dimethyl-3,4-dihydro-1H-quinolin-2-one. Eur J Med Chem 43:1730-1736.
23. Schoenebeck, F., et al. (2007) Reductive cleavage of sulfones and sulfonamides by a neutral organic super-electron-donor (S.E.D.) reagent. J Am Chem Soc 129:13368-13369.
24. Van Quaquebeke, E., et al. (2007) 2,2,2-Trichloro-N-({2-[2-(dimethylamino)ethyl]-1,3-dioxo-2,3-dihydro-1H-benzo[de]isoquinolin-5-yl}carbamoyl)acetamide (UNBS3157), a novel nonhematotoxic naphthalimide derivative with potent antitumor activity. J Med Chem 50:4122-4134.
25. Zee-Cheng, R. K., Cheng, C. C. (1985) N-(Aminoalkyl)imide antineoplastic agents. Synthesis and biological activity. J Med Chem 28:1216-1222.

1. An assay for identifying a compound that modifies miR-122 function that comprises a plasmid construct comprising a reporter gene, and miR-122 target sequence wherein the plasmid construct is in a cell that expresses miRNA-122 and exposing the cell to a compound and measuring a change in expression of the reporter gene.

2. The assay according to claim 1, wherein the reporter gene encodes luciferase.

3. A method for identifying a compound that modifies miR-122 function that comprises adding a compound to a cell that expresses miRNA-122 and containing a plasmid construct comprising a reporter gene and miR-122 target sequence and measuring a change in expression of the reporter gene.

4. The method according to claim 3, wherein the reporter gene encodes luciferase.

5. A compound selected from structures I, II and III 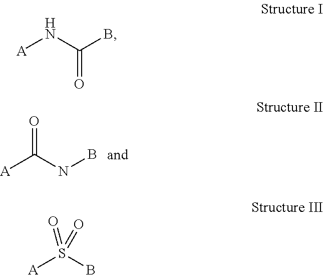
that modifies miR-122 function wherein A and B are independently selected from unsubstituted and substituted alkyl, alkenyl, alkynyl, cycloalkyl, alkoxy, acyl, aryl, aryloxy, heterocyclyl, heteroaryl, aralkyl, NO₂, and amino.

6. A compound that modifies miR-122 function selected from N-(Naphthalen-3-yl)benzamide, 4-Acetyl-N-(naphthalen-3-yl)benzamide and 3,4-dimethoxy-N-(naphthalen-6-yl)benzamide or a pharmaceutically acceptable salt, stereoisomer, tautomer or solvate thereof.

7. A compound that modifies miR-122 function selected from
N-(Naphthalen-2-yl)-4-nitrobenzamide;
2,4-Dichloro-N-phenylbenzamide;
N-(4-Aminophenyl)-2,4-dichlorobenzamide;
2,4-Dichloro-N-(4-iodophenyl)benzamide;
2,4-Dichloro-N-((naphthalen-6-yl)methyl)benzamide;
Ethyl-6-(3,4-dihydroquinolin-1(2H)-ylsulfonyl)-3,4-dihydroquinoline-1(2H)-carboxylate;
N-Phenyl-1,2,3,4-tetrahydroquinoline-6-sulfonamide;
6-(3,4-Dihydroquinolin-1(2H)-ylsulfonyl)-1,2,3,4-tetrahydroquinoline;
N-(4-Aminophenyl)-1,2,3,4-tetrahydroquinoline-6-sulfonamide;
2-(2-(Dimethylamino)ethyl)-1H-benzo[de] isoquinoline-1,3(2H)-dione;
5-Amino-2-(2-aminoethyl)-1H-benzo[de] isoquinoline-1,3(2H)-dione;
5-amino-2-(naphthalen-2-ylmethyl)-1H-benzo[de] isoquinoline-1,3(2H)-dione;
2-(2-(Dimethylamino)ethyl)-5,8-dinitro-1H-benzo[de]isoquinoline-1,3(2H)-dione and
compounds shown in FIGS. 10, 11 and 12 or a pharmaceutically acceptable salt, stereoisomer, tautomer or solvate thereof.

8. A method for treating liver cancer comprising administering to a subject in need thereof a compound of claim 5 that activates miR-122 function.

9. A method for treating hepatitis C virus infection comprising administering to a subject in need thereof a compound of claim 5 that inhibits miR-122 function.

10. (canceled)

11. A method of modulating miR-122 function in a cell comprising administering to the cell an amount of a compound as claimed in claim 5 in an amount sufficient to modulate the function of miR-122.

12. A method of modulating miR-122 function in a subject in need thereof cell comprising administering to the subject an amount of a compound as claimed in claim 5 sufficient to modulate the function of miR-122.

13. A method for treating liver cancer comprising administering to a subject in need thereof a compound of claim 6 that activates miR-122 function.

14. A method for treating liver cancer comprising administering to a subject in need thereof a compound of claim 7 that activates miR-122 function.

15. A method for treating hepatitis C virus infection comprising administering to a subject in need thereof a compound of claim 6 that inhibits miR-122 function.

16. A method for treating hepatitis C virus infection comprising administering to a subject in need thereof a compound of claim 7 that inhibits miR-122 function.

17. A method of modulating miR-122 function in a cell comprising administering to the cell an amount of a compound as claimed in claim 6 in an amount sufficient to modulate the function of miR-122.

18. A method of modulating miR-122 function in a cell comprising administering to the cell an amount of a compound as claimed in claim 7 in an amount sufficient to modulate the function of miR-122.

19. A method of modulating miR-122 function in a subject in need thereof cell comprising administering to the subject an amount of a compound as claimed in claim 6 sufficient to modulate the function of miR-122.

20. A method of modulating miR-122 function in a subject in need thereof cell comprising administering to the subject an amount of a compound as claimed in claim 7 sufficient to modulate the function of miR-122.
